(19) United States
(12) Patent Application Publication
(10) Pub. No.: US 2003/0236276 A1
(43) Pub. Date: Dec. 25, 2003

(54) Pyrrolo[2.1-a]isoquinoline derivatives

(76) Inventors: Ulrich Niewohner, Wermelskirchen (DE); Maria Niewohner, Wermelskirchen (DE); Marcus Bauser, Wuppertal (DE); Jens-Kerim Erguden, Wulfrath (DE); Dietmar Flubacher, Freiburg (DE); Paul Naab, Wuppertal (DE); Thorsten-Oliver Repp, Wesseling (DE); Jurgen Stoltefuss, Haan (DE); Nils Burkhardt, Dusseldorf (DE); Andrea Sewing, Broadstairs (GB); Michael Schauer, Wuppertal (DE); Karl-Heinz Schlemmer, Wuppertal (DE); Olaf Weber, Woodbridge, CT (US); Stephen J. Boyer, Fairfield, CT (US); Mark Miglarese, Ivorytown, CT (US); Jianmei Fan, Hamden, CT (US); Barton Phillips, New Haven, CT (US); Brian C. Raudenbush, Hamden, CT (US); Yamin Wang, Sandy Hook, CT (US)

Correspondence Address: JEFFREY M. GREENMAN, VICE PRESIDENT, PATENTS AND LICENSING, BAYER CORPORATION, 400 MORGAN LANE, WEST HAVEN, CT, 06516, US

(21) Appl. No.: 10/213,290

(22) Filed: Aug. 5, 2002

Related U.S. Application Data
(60) Provisional application No. 60/310,358, filed on Aug. 6, 2001.

(51) Int. Cl. A61K 31/4745; C07D 451/02

(52) U.S. Cl. 514/291; 546/94

(57) ABSTRACT

The present invention relates to pyrrolo[2.1-a]isoquinolines which are inhibitors of phosphodiesterase 10a, a process for preparing these compounds and a method of treating cancer in humans and animals by administering these compounds.

This application claims priority from U.S. Provisional Application No. 60/310,358, filed Aug. 6, 2001.

### BACKGROUND OF THE INVENTION

The present invention relates to pyrrolo[2.1-a]isoquinoline derivatives which are inhibitors of phosphodiesterase 10a, a process for preparing those compounds and a method of treating cancer in humans or animals by administering those compounds.

Cyclic AMP metabolism is regulated by the opposing activities of adenylyl cyclase, which generates cAMP in response to extracellular stimuli (e.g. engagement of G-protein coupled receptors by their cognate ligands), and 3′,5′-cyclic nucleotide phosphodiesterases (PDEs), which hydrolyze cAMP to 5′-AMP. Signal transduction via cAMP is associated with transcriptional events that can result in the inhibition of cellular proliferation (T. J. Shaw et al., Exp. Cell Res. 273, 95 (2002); T. W. Moody et al., Ann. N.Y. Acad. Sci. 921, 26 (2000); W. L. Lowe et al., Endocrinology 138, 2219 (1997); D. A. Albert, J. Clin. Invest. 95, 1490 (1995); M. I. Mednieks et al., FEBS Lett. 254, 83 (1989)). Indeed, elevation of intracellular cAMP concentration is growth inhibitory for several human tumor cell lines, including those derived from breast, lung and colorectal carcinomas (B. Wagner et al., Biochem. Pharmacol. 63, 659 (2002); S. B. Jakowlew et al., Peptides 21, 1831 (2000); I. S. Fentimen et al., Mol. Biol. Med. 2, 81 (1984); P. Cassoni et al., Int. J. Cancer 72, 340 (1997); S. Shafer et al., Biochem. Pharmacol. 56, 1229 (1998); N. M. Hoosein et al., Regul. Peptides 24, 15 (1989)). In several human breast carcinoma cell lines, increased cAMP production through stimulation of adenylate cyclase activity and/or reduction in cAMP catabolism through inhibition of phosphodiesterase activity has been shown to result in increased steady state levels of cAMP and growth inhibition (D. Melck et al., FEBS Letters 463, 235 (1999); N. Veber et al., Eur. J. Cancer 30A, 1352 (1994); J. A. Fontana et al., J. Natl. Cancer Inst. 78, 1107 (1987); T. A. Slotkin et al., Breast Cancer Res. and Treatment 60, 153 (2000)). In contrast to breast tumor cell lines, normal human mammary epithelial cells are stimulated to proliferate by elevation of intracellular cAMP (I. S. Fentimen et al., Mol. Biol. Med. 2, 81 (1984)). These observations suggest that elevation of intracellular cAMP may selectively inhibit breast tumor cell proliferation. Interestingly, it has been reported that neoplastic mammary tissues have higher levels of low-Km phosphodiesterase activity compared to normal breast tissue, suggesting that tumors may gain a growth or survival advantage by keeping intracellular cAMP levels in check (A. Larks Singer et al., Cancer Res. 36, 60 (1976)).

The ICAST (Inhibitor of Cyclic AMP Signal Transduction) gene encodes a specific 3′,5′-cyclic nucleotide phosphodiesterase. Compared to corresponding normal tissues, ICAST mRNA is overexpressed in breast carcinoma specimens, liver metastases of colorectal carcinoma and non-small cell lung carcinomas. The ICAST cDNA was also recently cloned by other groups and named PDE 10a (K. Fujishige et al., J. Biol. Chem. 274, 18 438 (1999); S. H. Soderling et al., Proc. Natl. Acad. Sci. USA 96, 7071 (1999); K. Loughney et al., Gene 234, 109 (1999)). Published expression data for ICAST mRNA show a very limited distribution across adult human tissues, with highest levels observed in the testis, caudate nucleus and putamen (K. Fujishige et al., 1999). Increased expression of ICAST mRNA in human tumor specimens indicates that ICAST may play an important role in tumor cell growth and/or survival under conditions of elevated cAMP generation. Selective inhibition of ICAST activity in tumor cells should lead to increased cAMP concentrations and growth inhibition. The expression profile of ICAST and the published reports indicating that breast, lung and colon carcinomas are particularly sensitive to elevation of intracellular cAMP indicate that ICAST may play critical roles specifically in those tumor types. In addition to elevation of cAMP, inhibition of ICAST activity should also decrease the intracellular concentration of 5-AMP, which could limit purine pools and DNA synthesis in rapidly dividing tumor cells.

Pyrrolo[2.1-a]isoquinoline derivatives of formula (A) are described in J. Med. Chem. 27, 1321 (1984) and in J. Med. Chem. 31, 2097 (1988):

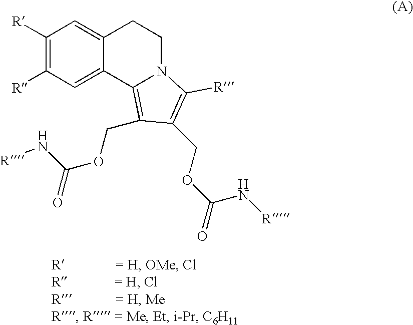

These compounds are described as having antineoplastic activity, which however is stated to be due to the carbamate moieties being electrophilic centers enabling the compounds (A) to react via an alkyl-oxygen cleavage mechanism. It is not mentioned that these compounds have any PDE 10a inhibitory activity.

Tetracyclic compounds of formula (B) containing a pyrrolo[2.1-a]isoquinoline moiety are described in Arch. Pharm. 321, 481 (1988):

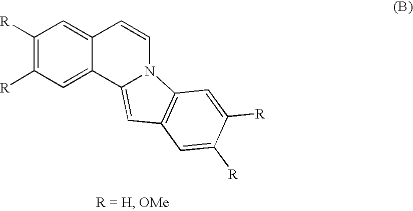

The compounds (B) are described as having anti-tumor activity due to their ability to intercalate into DNA. It is not mentioned that they have any PDE 10a inhibitory activity.

The synthesis of pyrrolo[2.1-a]isoquinoline derivatives of formula (C) is described in H. Meyer, Liebigs Ann. Chem. 9, 1534-1544 (1981):

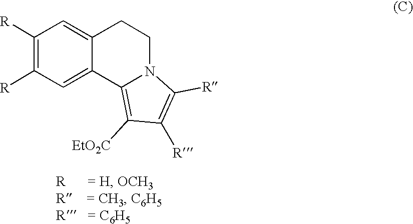

These compounds are not described as having any biological activity, and it is not mentioned that they have any PDE 10a inhibitor activity.

Compounds of the formula (D) are described in GB 1 153 670 A:

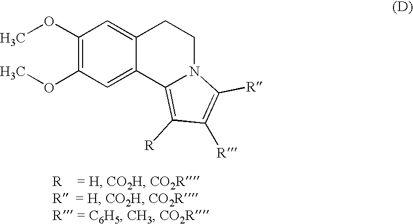

These compounds are described as having hypotensive, sympathicolytic and psychotropic properties, but it is not mentioned that they have any PDE 10a inhibitory activity.

The synthesis of compounds of the formula (E) is described in U.S. Pat. No. 4,694,085:

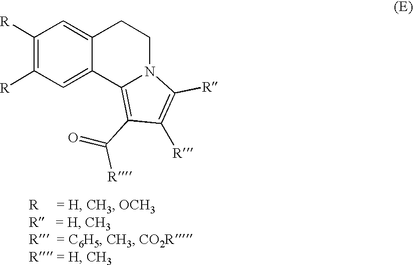

It is not mentioned that these compounds have any PDE 10a inhibitory activity.

Derivatives of the formula (F) are described in WO 98/55118:

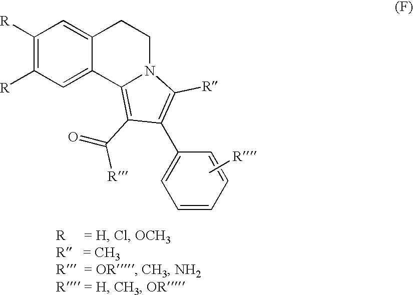

These compounds are described as useful for the treatment of diseases such as psoriasis. However, the compounds disclosed in WO 98/55118 are described as having virtually no cytotoxic activity; it is not mentioned that they have any PDE 10a inhibitor activity.

### BRIEF SUMMARY OF THE INVENTION

Surprisingly, it has been found that the pyrrolo[2.1-a]dihydroisoquinolines of the present invention inhibit PDE 10a and exhibit an antiproliferative activity.

The present invention relates to compounds of the formula

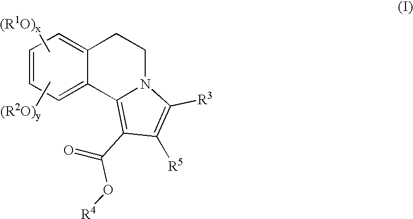

wherein

x and y independently from each other denote zero or 1;

R¹ and R² independently from each other denote hydrogen, C₁₋₄-alkyl or CF₃;

R³ and R⁴ independently from each other denote C₁₋₄-alkyl;

R⁵ denotes

i) C₁₋₁₂-alkyl, optionally having from 1 to 3 substituents selected from the group consisting of C-₁₋₆-alkoxy, C₆₋₁₀-aryl, and heteroaryl;

or

ii) C₃₋₈-cycloalkyl, optionally having from 1 to 3 substituents selected from the group consisting of C₁₋₆-alkyl, C-₁₋₆-alkoxy, COOR⁶, C₆₋₁₀-aryl, and heteroaryl;

or

iii) heteroaryl optionally substituted with up to 3 substituents selected from the group consisting of

a) C₁₋₆-alkyl, C-₁₋₆-alkoxy, C₆₋₁₀-aryl-C₁₋₆-alkyl, heteroaryl-C₁₋₆-alkyl, C₁₋₆-alkoxy-C₁₋₆-alkyl, C₁₋₆-alkoxy-C₁₋₆-alkoxy-C₁₋₆-alkyl, cyano-C₁₋₆-alkyl, and C₆₋₁₀-aryl (each of which can optionally be substituted by halogen up to perhalo),

b) COR⁶,

c) COOR⁶,

d) hydroxyl,

e) halogen,

f) cyano,

g) SO₂R⁶, and

h) saturated 5- to 9-membered nitrogen-containing heterocyclyl (which saturated heterocyclyl may contain up to 2 further heteroatoms selected from the group consisting of N, O and S and which saturated heterocyclyl can be further substituted with one or more radicals selected from the group consisting of hydroxyl, NH₂, C₁₋₆-alkyl, C₁₋₆-alkoxy and C₆₋₁₀-aryl);

wherein R⁶ denotes

1) hydrogen,

2) C₁₋₆-alkyl optionally substituted with halogen up to perhalo,

3) C₃₋₈-cycloalkyl,

4) C₆₋₁₀-aryl optionally substituted with C₁₋₆-alkoxy,

5) heteroaryl-C₁₋₆-alkyl,

6) C₆₋₁₀-aryl-C₁₋₆-alkyl optionally substituted with up to 2 C₁₋₆-alkoxy, or

7) —NR⁷R⁸, wherein (i) R⁷ and R⁸ are each independently selected from the group consisting of hydrogen, C₁₋₆-alkyl, C₃₋₈-cycloalkyl, C₁₋₈-heterocyclyl, and C₆₋₁₀-aryl optionally substituted with C₁₋₆-alkoxy, or

(ii) R⁷ and R⁸ together with the nitrogen atom to which they are attached form a 5- to 7-membered heterocyclyl which may contain up to 2 further heteroatoms selected from the group consisting of N, O, and S, which heterocyclyl can further be substituted with 1 to 3 radicals selected from the group consisting of OH, C₁₋₄-alkyl, C₁₋₄-alkoxy, C₆₋₁₀-aryl, and heteroaryl;

or

iv) phenyl fused to a 5- to 7-membered saturated cycloalkyl optionally containing up to two hetero atoms selected from the group consisting of O, N, and S, with the proviso that said two heteroatoms cannot both be O, optionally substituted with 1-3 substituents selected from the group consisting of hydroxy, halogen, C₁₋₆-alkyl, C₁₋₆-alkoxy, C₁₋₆-alkylsulfonyl, phenylsulfonyl, N-C₁₋₆-alkylcarboxamido, N-(C₃₋₈-cycloalkyl)-carboxamido, N-phenylcarboxamido, N-(C₁₋₆-alkoxy-phenyl)-carboxamido; and (C₁₋₆-alkyl)-carbonyl wherein said C₁₋₆-alkyl)-carbonyl may optionally be substituted by halogen up to perhalo;

and an isomer, a pharmaceutically acceptable salt, a hydrate, or a hydrate of a pharmaceutically acceptable salt thereof.

An alternative embodiment of the present invention relates to compounds of the formula (I),

wherein

x and y independently from each other denote zero or 1;

R¹ and R² independently from each other denote hydrogen, C₁₋₄-alkyl or CF₃;

R³ and R⁴ independently from each other denote C₁₋₄-alkyl;

R⁵ denotes

i) C₁₋₁₂-alkyl, optionally having 1 to 3 substituents selected from the group consisting of C-₁₋₆-alkoxy and C₆₋₁₀-aryl;

or

ii) C₃₋₈-cycloalkyl;

or

iii) heteroaryl optionally substituted with up to 3 substituents selected from the group consisting of

a) C₁₋₆-alkyl, C-₁₋₆-alkoxy, C₆₋₁₀-aryl-C₁₋₆-alkyl, heteroaryl-C₁₋₆-alkyl, C₁₋₆-alkoxy-C₁₋₆-alkyl, cyano-C₁₋₆-alkyl, C₁₋₆-alkoxy-C₁₋₆-alkoxy-C₁₋₆-alkyl, and C₆₋₁₀-aryl (each of which can optionally be substituted with halogen radicals up to perhalo),

b) COR⁶,

c) COOR⁶,

d) hydroxyl,

e) halogen,

f) cyano,

g) SO₂R⁶, and

h) saturated 5- to 9-membered nitrogen-containing heterocyclyl (which saturated heterocyclyl may contain up to 2 further hetero atoms selected from the group consisting of N, O and S and which saturated heterocyclyl can be further substituted with one or more radicals selected from the group consisting of hydroxyl, NH₂, C₁₋₆ alkyl, C₁₋₆-alkoxy, and C₆₋₁₀-aryl);

wherein R⁶ denotes

1) hydrogen,

2) C₁₋₆-alkyl optionally substituted with halogen up to perhalo,

3) C₃₋₈-cycloalkyl,

4) C₆₋₁₀-aryl optionally substituted with C₁₋₆-alkoxy,

5) heteroaryl-C₁₋₆-alkyl,

6) C₆₋₁₀-aryl-C₁₋₆-alkyl optionally substituted with up to 2 C₁₋₆-alkoxy, or

7) —NR⁷R⁸ wherein R⁷ and R⁸ are each independently selected from the group consisting of hydrogen, C₁₋₆-alkyl, C₃₋₈-cycloalkyl, heterocyclyl, and C₆₋₁₀-aryl which C₆₋₁₀-aryl is optionally substituted with C₁₋₆-alkoxy;

or

iv) indolinyl optionally substituted with up to three substitutents selected from the group consisting of hydrogen, halogen, C₁₋₆-alkyl, C₁₋₆-alkoxy, C₁₋₆-alkylsulfonyl, phenylsulfonyl, N-C₁₋₆-alkylcarboxamido, N-(C₃₋₈-cycloalkyl)-carboxamido, N-phenylcarboxamido, N-(methoxyphenyl)-carboxamido, and (C₁₋₆-alkyl)-carbonyl wherein said (C₁₋₆-alkyl)-carbonyl may optionally be substituted by halogen up to perhalo,

and an isomer, a pharmaceutically acceptable salt, a hydrate, or a hydrate of a pharmaceutically acceptable salt thereof.

A further alternative embodiment of the present invention relates to compounds of the formula (I),

wherein

x and y each denote 1;

R¹ and R² independently from each other denote hydrogen or C₁₋₄-alkyl;

R³ and R⁴ independently from each other denote C₁₋₄-alkyl;

R⁵ denotes

i) methyl, ethyl, n-propyl, iso-propyl, n-butyl, n-pentyl, n-hexyl, n-heptyl, 2-phenylpropyl, 2-butyl, benzyl;

or

ii) cyclopropyl, cyclopentyl or cyclohexyl;

or

iii) a) thienyl optionally substituted with Cl, Br, I, C₁₋₆-alkyl;

b) pyrrolyl optionally substituted with Cl, Br, I, C₁₋₆-alkyl;

c) furyl optionally substituted with Cl, Br, I, C₁₋₆-alkyl;

d) thiazolyl optionally substituted with Cl, Br, I, C₁₋₆-alkyl;

e) imidazolyl optionally substituted with Cl, Br, I, C₁₋₆-alkyl;

f) pyridyl optionally substituted with Cl, Br, I;

g) pyrimidinyl optionally substituted with pyrrolidine;

h) indazolyl optionally substituted with 2-fluorobenzyl;

i) benzimidazolyl;

j) benzoxazolyl

k) quinolinyl optionally substituted with hydroxyl, methyl or phenyl; or

l) indolyl optionally substituted with up to three substituents selected from the group consisting of F, Cl, Br, I, C₁₋₆-alkyl, C₁₋₆-alkoxy-C₁₋₆-alkyl, C₁₋₆-alkoxy-C₁₋₆-alkoxy-C₁₋₆-alkyl, cyano-C₁₋₆-alkyl, C₁₋₆-alkoxy, benzyl, fluorobenzyl, pyridylmethyl, phenylsulfonyl, formyl, (C₁₋₆-alkyl)-carbonyl, (C₃₋₈-cycloalkyl)-carbonyl, phenylcarbonyl, methoxyphenylcarbonyl, and dimethoxybenzylcarbonyl;

or

(iv) indolinyl optionally substituted with up to three substituents selected from the group consisting of C₁₋₆-alkylsulfonyl, phenylsulfonyl, N-C₁₋₆-alkylcarboxamido, N-(C₃₋₈-cycloalkyl)-carboxamido, N-phenylcarboxamido, N-(methoxyphenyl)-carboxamido, and (C₁₋₆-alkyl)-carbonyl wherein said (C₁₋₆-alkyl)-carbonyl may optionally be substituted by halogen up to perhalo,

and an isomer, a pharmaceutically acceptable salt, a hydrate, or a hydrate of a pharmaceutically acceptable salt thereof.

Compounds (I) wherein the radicals (R¹O)ₓ and (R²O)_{y} are attached to the phenyl ring in the following positions, are preferred:

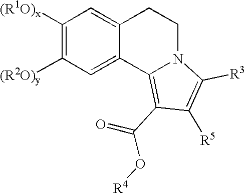

Furthermore, according to the present invention the respective 5,6-dihydropyrrolo derivatives of formula (I) are preferred.

Furthermore, the compounds of Examples 1, 11, 23, 46, 47, 49, 52, 54, 60, 65, 80, and 86 are particularly preferred.

### DETAILED DESCRIPTION OF THE INVENTION

Physiologically acceptable salts according to the invention are non-toxic salts which in general are accessible by reaction of the compounds (I) with an inorganic or organic base or acid conventionally used for this purpose. Non-limiting examples of physiologically acceptable salts of compounds (I) include the alkali metal salts, e.g. lithium, potassium and sodium salts, the alkaline earth metal salts such as the magnesium or calcium salts, the quaternary ammonium salts such as, for example, the triethyl ammonium salts, acetates, benzenesulphonates, benzoates, dicarbonates, disulphates, ditartrates, borates, bromides, carbonates, chlorides, citrates, dihydrochlorides, fumarates, gluconates, glutamates, hexylresorcinates, hydrobromides, hydrochlorides, hydroxynaphthoates, iodides, isothionates, lactates, laurates, malates, maleates, mandelates, mesylates, methylbromides, methylnitrates, methylsulphates, nitrates, oleates, oxalates, palmitates, pantothenates, phosphates, diphosphates, polygalacturonates, salicylates, stearates, sulphates, succinates, tartrates, tosylates and valerates, and other salts used for medicinal purposes.

The present invention includes both the individual enantiomers or diastereomers and the corresponding racemates, diastereomer mixtures and salts of the compounds according to the invention. In addition, all possible tautomeric forms of the compounds described above are included according to the present invention. The diastereomer mixtures can be separated into the individual isomers by chromatographic processes. The racemates can be resolved into the respective enantiomers either by chromatographic processes on chiral phases or by resolution.

In the context of the present invention, the substituents, if not stated otherwise, in general have the following meaning:

Alkyl per se as well as the prefixes “alkyl” and “alk” in the terms “alkylcarbonyl”, “alkylsulphonyl”, “alkylaminocarbonylamino”, “alkoxy” and “alkoxycarbonyl” represent a linear or branched alkyl radical preferably having 1 to 12, more preferably 1 to 6 carbon atoms. Non-limiting examples of alkyl radicals include methyl, ethyl, propyl, isopropyl, butyl, isobutyl, pentyl, isopentyl, hexyl, and isohexyl.

Non-limiting examples of alkylcarbonyl radicals include acetyl, ethylcarbonyl, propylcarbonyl, isopropylcarbonyl, butylcarbonyl and isobutylcarbonyl. The terms “alkylcarbonyl” and “acyl” are used synonymously.

Non-limiting examples of alkylsulphonyl radicals include methylsulphonyl, ethylsulphonyl, propylsulphonyl, isopropylsulphonyl, butylsulphonyl and isobutylsulphonyl.

Non-limiting examples of alkylaminocarbonylamino radicals include methylaminocarbonylamino, ethylaminocarbonylamino, propylaminocarbonylamino, isopropylaminocarbonylamino, butylaminocarbonylamino and isobutylaminocarbonylamino.

Non-limiting examples of alkoxy radicals include methoxy, ethoxy, propoxy, isopropoxy, butoxy, isobutoxy, pentoxy, isopentoxy, hexoxy, isohexoxy. The terms “alkoxy” and “alkyloxy” are used synonymously.

Non-limiting examples of alkoxycarbonyl include methoxycarbonyl, ethoxycarbonyl, propyloxycarbonyl, isopropyloxycarbonyl, butyloxycarbonyl and isobutyloxycarbonyl.

. . . alkyl in the term “aryl-alkyl” represents a linear or branched (bivalent) alkylene radical preferably having 1 to 4 carbon atoms. Non-limiting examples include methylene, 1,2-ethylene, 1,2- and 1,3-propylene, and 1,2-, 1,3-, 1,4- and 2,3-butylene; methylene is preferred.

Alkylene represents a linear or branched (bivalent) alkylene radical preferably having 1 to 4 carbon atoms. Non-limiting examples of alkylene radicals include methylene, ethylene, propylene, α-methylethylene, β-methylethylene, α-ethylethylene, β-ethylethylene, butylene, α-methylpropylene, β-methylpropylene, and γ-methylpropylene.

Cycloalkyl represents a saturated cycloalkyl radical preferably having 3 to 8 carbon atoms. Non-limiting examples of cycloalkyl radicals include cyclopropyl, cyclopentyl, cyclohexyl, cycloheptyl and cyclooctyl; cyclopropyl, cyclopentyl and cyclohexyl are preferred.

Aryl per se and in the terms “aryloxy”, “aryl-alkyl” and “arylaminocarbonylamino” represents an aromatic radical preferably having 6 to 14, more preferably 6 to 10 carbon atoms. Non-limiting examples of aryl radicals include phenyl, naphthyl and phenanthrenyl; non-limiting examples of aryloxy radicals include phenyloxy; non-limiting examples of aryl-alkyl radicals include benzyl; non-limiting examples of arylaminocarbonylamino radicals include phenylaminocarbonylamino, benzylaminocarbonyl amino, naphthylaminocarbonylamino, and phenanthrenylaminocarbonylamino.

Heterocyclyl in the context of the invention represents a saturated, partially unsaturated or aromatic preferably 3- to 9-membered ring or ring system containing at least one carbon atom and containing at least 1, up to 4, heteroatoms from the group consisting of S, N, and O, which ring or ring system can be linked via a carbon atom or a nitrogen atom, if such an atom is present. Non-limiting heterocyclyl examples include oxadiazolyl, thiadiazolyl, pyrazolyl, pyridyl, pyrimidinyl, pyridazinyl, pyrazinyl, quinolinyl, isoquinolinyl, indolyl, thienyl, furyl, pyrrolyl, N-methylpyrrolyl, indazolyl, benzimidazolyl, benzoxazolyl, pyrrolidinyl, piperazinyl, tetrahydropyranyl, tetrahydrofuranyl, 1,2,3-triazolyl, thiazolyl, oxazolyl, indolinyl, imidazolyl, morpholinyl, thiomorpholinyl, piperidyl, or aziridyl. Preferred examples include thiazolyl, furyl, oxazolyl, thienyl, pyrazolyl, imidazolyl, 1,2,3-triazolyl, pyridyl, pyrrolyl, pyrimidinyl, pyridazinyl tetrahydropyranyl, indolyl, indazolyl, benzimidazolyl, quinolinyl, benzoxazolyl, and indolinyl.

Heteroaryl denotes an aromatic heterocyclyl (for “heterocyclyl”, see paragraph above). In fused ring systems which contain hetero atoms as ring members, “heteroaryl” necessarily comprises at least one aromatic hetero ring. Non-limiting examples include thiazolyl, furyl, oxazolyl, thienyl, pyrazolyl, imidazolyl, 1,2,3-triazolyl, pyridyl, pyrrolyl, pyrimidinyl, pyridazinyl tetrahydropyranyl, indolyl, indazolyl, benzimidazolyl, quinolinyl, and benzoxazolyl. Indolinyl is not considered as a “heteroaryl” for the purposes of this invention.

Halogen in the context of the invention represents fluorine, chlorine, bromine and iodine.

The present invention also relates to a process for manufacturing the compounds according to the invention comprising the reaction of a compound of the formula

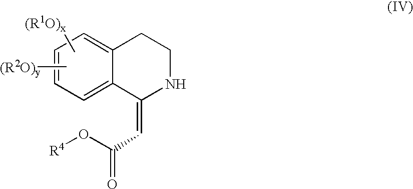

wherein x, y, R¹, R² and R⁴ are as defined above,

[A] with the compounds of the formulae

R⁵—CHO (II) and R³—CH₂—NO₂ (III)

wherein R³and R⁵ are as defined above, or

[B] with a compound of the formula

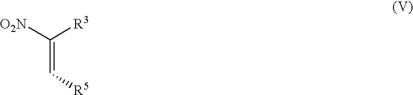

wherein R³ and R⁵ are as defined above, and optionally

[C] the conversion of the compound obtained through either process [A] or [B] into an isomer, a pharmaceutically acceptable salt, a hydrate, or a hydrate of a pharmaceutically acceptable salt thereof.

The compounds (II) are commercially available or can be synthesized according to methods commonly known to those skilled in the art (I. T. Harrison and S. Harrison, Compendium of Organic Synthetic Methods, pp. 132-176, Wiley-Interscience; T. D. Harris and G. P. Roth, J. Org. Chem. 44, 146 (1979); E. Muiller (ed.), “Methoden der Organischen Chemie” [Methods of Organic Chemistry] (Houben-Weyl), Vol. VII/1 Sauerstoff-Verbindungen II, Georg Thieme Verlag, Stuttgart 1954).

The compounds (III) are commercially available.

The compounds (IV) can be synthesized by reacting compounds of the formula

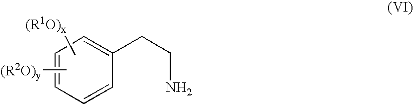

wherein x, y, R¹ and R² are as defined above,

with compounds of the formula

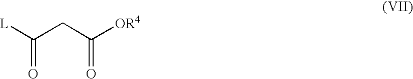

wherein

R⁴ is as defined above and

L is a leaving group, for example a halogen radical such as Cl, or a radical of the formula

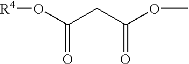

to give compounds of the formula

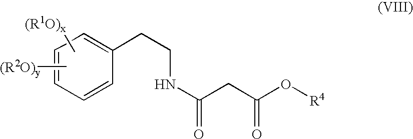

wherein x, y, R¹, R² and R⁴ are as defined above,

and reacting compound (VIII) with a dehydrating agent.

The compounds (VI) are commercially available or can be synthesized according to methods commonly known those skilled in the art (H. Mayer et al., Heterocycles 31, 1035 (1990); E. Müller (Ed.), “Methoden der Organischen Chemie” [Methods of Organic Chemistry] (Houben-Weyl), Vol. 11/1 Stickstoff-Verbindungen, Georg Thieme Verlag, Stuttgart 1957).

The compounds (VII) are commercially available or can be synthesized according to methods commonly known those skilled in the art [e.g. via acylation of acetic acid with an alkyl chloroformate or dialkyl carbonate (March, Advanced Organic Chemistry, 3^{rd} ed., p. 440-441, Wiley 1985) and converting the resultant monoester of malonic acid into e.g. the corresponding acid chloride or anhydride by methods commonly known those skilled in the art (see e.g. March, Advanced Organic Chemistry, 3^{rd} ed., p. 355, 388, Wiley 1985)].

The reaction between the compounds (VI) and (VII) is preferably carried out in a solvent. Suitable solvents comprise the customary organic solvents which are inert under the reaction conditions. Non-limiting examples include ethers, such as diethyl ether, dioxane, tetrahydrofuran, 1,2-dimethoxy ethane; hydrocarbons such as benzene, toluene, xylene, hexane, cyclohexane or mineral oil fractions; halogenated hydrocarbons such as dichloromethane, trichloromethane, carbon tetrachloride, dichloroethane, trichloroethylene or chlorobenzene; ketones such as acetone; esters such as ethyl acetate; nitrites such as acetonitrile; heteroaromatics such as pyridine; optionally N-alkylated carboxylic acid amides such as dimethyl formamide and dimethyl acetamide; alkyl sulfoxides such as dimethyl sulfoxide; optionally alkylated phosphoric acid amides such as hexamethylphosphoric acid tris-amide; and mixtures of the above-mentioned solvents. Dichloromethane is particularly preferred.

The compound (VII) is generally employed in an amount of from 1 to 4 mol per mol of compound (VI); an equimolar amount or slight excess of compound (VII) is preferred.

The reaction between the compounds (VI) and (VII) is preferably carried out in the presence of a base. Non-limiting examples include alkali metal hydrides and alkali metal alkoxides such as, for example, sodium hydride and potassium tert.-butoxide; C₁₋₄-alkyl amines such as, for example, triethyl amine; cyclic amines such as, for example, piperidine, pyridine, dimethylaminopyridine and—preferably—1,8-diazabicyclo[4.3.0]-undec-7-ene (DBU). The base is generally employed in an amount of from 1 to 4 mol per mol of compound (VI); an equimolar amount or slight excess of the base is preferred.

The reaction of the compounds (VI) and (VII) can be carried out within a relatively wide temperature range. In general, the reaction is carried out within a range of from 0 to 200° C., preferably from 0 to 70° C., and more preferably at room temperature.

For the cyclization of the compounds (VIII) to yield compounds (IV), dehydrating agents such as, for example, P₂O₅, POCl₃, and methane sulfonic anhydride are generally employed in an amount of from 1 to 10 mol, preferably from 1 to 2 mol of methane sulfonic anhydride or 4 to 8 mols of P₂O₅ and POCl₃, respectively, per mol of compound (VIII) in each case.

The cyclization reaction of the compounds (VIII) to yield the compounds (IV) is also preferably carried out in a solvent. Non-limiting examples comprise the customary organic solvents which are inert under the reaction conditions. They preferably include ethers such as diethyl ether, dioxane, tetrahydrofuran, 1,2-dimethoxy ethane; hydrocarbons such as benzene, toluene, xylene, hexane, cyclohexane and mineral oil fractions; halogenated hydrocarbons such as dichloromethane, trichloromethane, carbon tetrachloride, dichloroethane, trichloroethylene and chlorobenzene; esters such as ethyl acetate; ketones such as acetone; nitriles such as acetonitrile; heteroaromatics such as pyridine; optionally N-alkylated carboxylic acid amides such as dimethyl formamide and dimethyl acetamide; alkyl sulfoxides such as dimethyl sulfoxide; optionally alkylated phosphoric acid amides such as hexamethyl phosphoric acid trisamide; and mixtures thereof. Toluene is preferred if the reaction is carried out with P₂O₅ or methane sulfonic anhydride; and acetonitrile is preferred if the reaction is carried out with POCl₃ (Benovsky, Stille, Tetrahedron Lett. 38, 8475-8478 (1997)).

The temperature for the cyclization reaction of compounds (VIII) is preferably within a range of from 60 to 200° C., and more preferably within a range of from 80 to 120° C.

The above process steps are generally carried out under atmospheric pressure. However, it is also possible to carry them out under superatmospheric pressure or under reduced pressure (for example, in a range of from 0.5 to 5 bar). The reaction time can generally be varied within a relatively wide range. In general, the reaction is finished after a period of from 2 to 24 hours, preferably from 6 to 12 hours.

The compounds (V) are commercially available or can be synthesized according to the reaction of the compounds (II) and (III) described below (in the absence of the compound (IV)).

The reaction of compound (fV) with either compounds (II) and (III) or with compound (V) can be carried out as a one-pot synthesis, preferably in a solvent. Suitable solvents comprise the customary organic solvents which are inert under the reaction conditions. Non-limiting examples include ethers such as diethyl ether, dioxane, tetrahydroftiran, 1,2-dimethoxy ethane; hydrocarbons such as benzene, toluene, xylene, hexane, cyclohexane and mineral oil fractions; halogenated hydrocarbons such as dichloromethane, trichloromethane, carbon tetrachloride, dichloroethane, trichloroethylene and chlorobenzene; alcohols such as methanol, ethanol, n-propanol and isopropanol; esters such as ethyl acetate; ketones such as acetone; nitriles such as acetonitrile; heteroaromatics such as pyridine; optionally N-alkylated carboxylic acid amides such as dimethyl formamide and dimethyl acetamide; alkyl sulfoxides such as dimethyl sulfoxide; optionally alkylated phosphoric acid amides such as hexamethyl phosphoric acid trisamide; and mixtures thereof. Ethanol/isopropanol (approximately 1:1 vol/vol) mixtures are preferred.

The compounds (III) are generally employed in an amount of from 1 to 3 mol per mol of compound (II); an equimolar amount or slight excess of compound (III) is preferred.

The compounds (IV) are generally employed in an amount of from 0,1 to 1 mol, preferably from 0,3 to 1 mol, in each case per mol of compounds (II).

The reaction of compound (IV) with either compounds (II) and (III) or with compound (V) is preferably carried out in the presence of a base. Non-limiting examples include alkali metal hydrides and alkali metal alkoxides such as, for example, sodium hydride and potassium tert.-butoxide, C₁₋₄-alkyl amines such as, for example, triethyl amine; cyclic amines such as, for example, pyridine, dimethylaminopyridine, 1,8-diazabicyclo-[4.3.0]undec-7-ene (DBU) and—preferably—piperidine. The base is generally employed in an amount of from 0,1 to 1 mol, preferably from 0,3 to 1 mol, per mol of compound (II) or compound (V), respectively.

The reactions of compound (IV) with either compounds (II) and (III) or with compound (V) are generally carried out within a relatively wide temperature range. In general, they are carried out in a range of from −20 to 200° C., preferably from 0 to 100° C., and most preferably from 50 to 90° C. The steps of this reaction are generally carried out under atmospheric pressure. However, it is also possible to carry them out under super-atmospheric pressure or under reduced pressure (for example, in a range of from 0.5 to 5 bar). The reaction time can generally be varied within a relatively wide range. In general, the reaction is finished after a period of from 2 to 24 hours, preferably from 6 to 12 hours.

The compounds (V) are commercially available or can be synthesized in analogy to the reaction of compounds (II) and (III) described above (in the absence of compound (IV)).

The process according to the present invention can be illustrated by the following scheme:

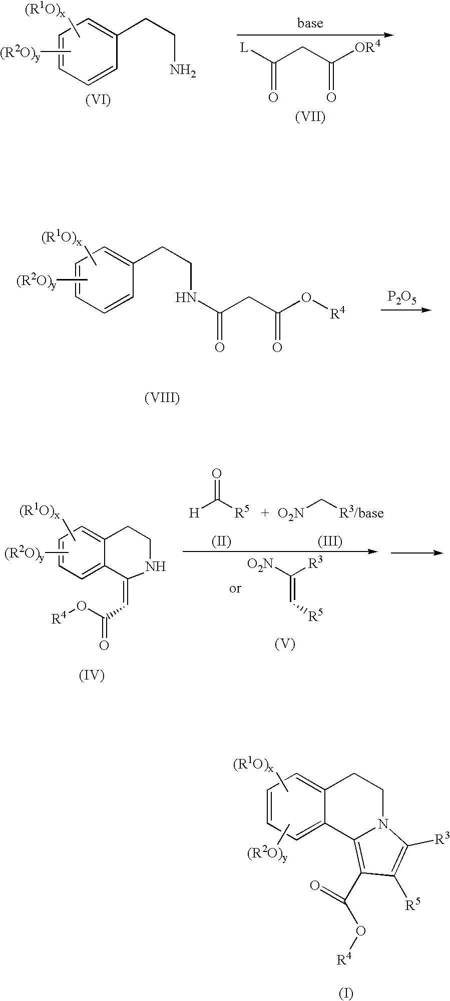

wherein x, y, R¹ to R⁵ and L are as defined above.

If the compounds (I) are not directly obtained by reacting the compounds (II), (III) and (IV) or (IV) and (V), the compounds thus obtained have to be converted into the compounds (I) by further reactions known to the man skilled in the art.

For example, compounds (I) wherein R⁵ is indolinyl can be further derivatized at the position-1 nitrogen to furnish a urea group by reaction with an isocyanate such as an alkyl or aryl isocyanate, in a suitable organic solvent, for example in a halogenated alkane such as dichloromethane, under conditions known to those skilled in the art.

Compounds (I) wherein R⁵ is indolinyl or indolyl can be further derivatized at the position-1 nitrogen to furnish alkylated, acylated, or sulfonylated products by reaction with an electrophile, for example diethyl sulfate, acetyl chloride, or benzenesulfonyl chloride, in a suitable organic solvent, for example in a halogenated alkane such as dichloromethane. These reactions are commonly conducted in the presence of a base, such as aqueous sodium hydroxide or triethyl amine, under conditions known to those skilled in the art.

Compounds (I) wherein R⁵ is indolyl can be further derivatized at the position-3 carbon to provide acyl adducts by reaction with an acid chloride, for example benzoyl chloride, in a suitable organic solvent, for example toluene. These reactions are commonly conducted in the presence of a Lewis acid, for example tin(II) chloride, under conditions known to those skilled in the art.

Compounds (I) wherein R⁵ is indolyl can be further derivatized at the position-3 carbon to provide formyl adducts by reaction with dimethyl formamide and a halogenating agent such as phosphorous oxychloride, in a suitable organic solvent, for example in a halogenated alkane such as dichloromethane, under conditions known to those skilled in the art.

The compounds of the present invention are inhibitors of phosphodiesterase 10a (PDE 10a). As outlined above, the inhibition of PDE 10a is a promising approach for the treatment of cancer. The biological tests described below show that the compounds (I) exhibit a pronounced anti-proliferation activity against tumor cells; they are therefore useful for the treatment of cancer. Furthermore, our investigations showed that they are also useful for treatment of conditions of pain and/or for the lowering of the temperature of the body in fever conditions.

The compounds according to the invention can be used as active ingredients for the production of medicaments against carcinomatous disorders. For this purpose, they can be converted into the customary formulations such as tablets, coated tablets, aerosols, pills, granules, syrups, emulsions, suspensions and solutions using inert, non-toxic, pharmaceutically suitable excipients or solvents. Preferably, the compounds according to the invention are used in an amount such that their concentration is approximately from 0.5 to 90% by weight, based on the ready-to-use formulations, the concentration, inter alia, being dependent on the corresponding indication of the medicament.

The formulations can be produced, for example, by extending the active compounds with solvents and/or excipients having the above properties, where, if appropriate, additionally emulsifiers or dispersants and, in the case of water as the solvent, an organic solvent can additionally be added.

Administration can be carried out in a customary manner, preferably orally, transdermally or parenterally, for example perlingually, buccally, intravenously, nasally, rectally or inhalationally.

For human use, in the case of oral administration, it is recommended to administer doses of from 0.001 to 50 mg/kg, preferably from 0.01 to 20 mg/kg. In the case of parenteral administration such as, for example, intravenously or via mucous membranes nasally, buccally or inhalationally, it is recommended to use doses of 0.001 to 0.5 mg/kg.

If appropriate, it may be necessary to depart from the amounts mentioned, namely depending on the body weight or the type of administration route, on the individual response towards the medicament, the manner of its formulation and the time or interval at which administration takes place. Thus, in some cases it may be sufficient to manage with less than the above mentioned minimum amount, while in other cases the upper limit mentioned must be exceeded. In the case of the administration of relatively large amounts, it may be recommended to divide these into several individual doses over the course of the day.

The compounds according to the invention are also suitable for use in veterinary medicine; they can be administered in a suitable formulation in accordance with general veterinary practice. Depending on the kind of animal to be treated, the veterinary surgeon can determine the nature of use and the dosage.

The present invention provides compounds for the use in a medicinal application, in particular for combating cancer.

The invention also provides a method of manufacturing a pharmaceutical composition by combining at least one of the compounds of the invention with at least one pharmaceutically acceptable formulating agent.

The invention further provides a pharmaceutical composition comprising as an active ingredient an effective amount of at least one of the compounds of the invention and at least one pharmaceutical active ingredient which is different from the compounds of the invention.

The invention further provides a medicament in dosage unit form comprising an effective amount of a compound according to the invention together with an inert pharmaceutical carrier.

The invention further provides a method of combating cancer in humans and animals comprising the administration of an effective amount of at least one compound according to the invention either alone or in admixture with a diluent or in the form of a medicament.

The invention further provides the use of at least one of the compounds of the invention for manufacturing a pharmaceutical composition for combating cancer.

The percentages in the description above, in the following tests and in the Examples are—if not stated otherwise—percentages by weight; parts are parts by weight. Solvent ratios, dilution ratios and concentrations in solutions of liquids in liquids are ratios and concentrations by volume.

Biological Tests

In vitro Enzyme Inhibition Assay:

Full-length recombinant PDE 10a was expressed in Sf9 insect cells (Invitrogen, Carlsbad, Calif., U.S.A.) using the Bac-to-Bac™ Baculovirus Expression System (Life Technologies, Gaithersburg, Md., U.S.A.). 48 hours post infection, cells were harvested and resuspended in 20 mL (per 1L culture) Lysis Buffer (50 mM Tris-HCl, pH 7.4, 50 mM NaCl, 1 mM MgCl₂, 1.5 mM EDTA, 10% glycerol plus 20 μL Protease Inhibitor Cocktail Set III [CalBiochem, La Jolla, Calif., U.S.A.]). Cells were sonicated at 4° C. for 1 minute and centrifuged at 10,000 RPM for 30 minutes at 4° C. Supernatant was removed and stored at −20° C. for activity assays.

The test compounds were serially diluted in DMSO using two-fold dilutions to stock concentrations ranging typically from 200 μM to 1.6 μM (final concentrations in the assay range from 4 μM to 0.032 μM). 96-well assay isoplates (Wallac Inc., Atlanta, Ga., U.S.A.) were loaded with 2 μL of the serially diluted individual test compounds followed by 50 μL of a dilution of crude recombinant PDE 10a-containing sf9 cell lysate. The dilution of the lysate was selected such that less than 70% of the substrate is converted during the later incubation (typical dilution: 1:10000; dilution buffer: 50 mM Tris/HCl pH 7.5, 8.3 mM MgCl₂, 1.7 mM EDTA, 0.2% BSA). The substrate, [5′,8-³H] adenosine 3′,5′-cyclic phosphate (1 μCi/μL; Amersham Pharmacia Biotech., Piscataway, N.J., U.S.A.), was diluted 1:2000 in assay buffer (assay buffer: 50 mM Tris/HCl pH 7.5, 8.3 mM MgCl₂, 1.7 mM EDTA) to give a final working concentration of 0.0005 μCi/μL. The enzymatic assay was initiated by addition of 50 μL (0.025 μCi) of diluted substrate. Reactions were incubated at room temperature for 60 minutes and terminated by addition of 25 μL of 18 mg/mL Yttrium Scintillation Proximity Beads (Amersham Pharmacia Biotech., Piscataway, N.J., U.S.A.). Plates were sealed and incubated at room temperature for 60 minutes. Plates were read for 30 seconds/well using a Microbeta counter (Wallac Inc., Atlanta, Ga., U.S.A.). The IC₅₀ values were determined by plotting compound concentration versus percent inhibition. Representative results are shown in Table 1:

TABLE 1

| Example No. | IC₅₀ (nM) |
|---|---|
| 5 | 370 |
| 7 | 140 |
| 8 | 94 |
| 12 | 365 |
| 23 | 71 |
| 46 | 220 |
| 49 | 110 |
| 50 | 330 |
| 52 | 220 |
| 56 | 68 |
| 57 | 1500 |
| 61 | 200 |
| 66 | 90 |
| 71 | 170 |
| 76 | 150 |
| 81 | 100 |
| 87 | <30 |

In vitro Proliferation Inhibition Assay:

MDA-MB-231 human breast carcinoma cells (ATCC #HTB26) were cultured in standard growth medium (DMEM), supplemented with 10% heat-inactivated FBS, 10 mM HEPES, 2 mM glutamine, 100 U/mL penicillin, and 100 μg/mL streptomycin) at 37° C. in 5% CO₂ (vol/vol) in a humidified incubator. Cells were plated at a density of 3000 cells per well in 100 μL growth medium in a 96 well culture dish. 24 hours after plating, lactate dehydrogenase (LDH) activity was determined using the Cytotox 96 Non-radioactive Cytotoxicity Kit (Promega, Madison Wis., U.S.A.) to yield T₀ₕ LDH values. Briefly, cells were lysed with the addition of 200 μL of Lysis Buffer (included in the Promega Kit) and lysates were further diluted 1:50 in Lysis Buffer. 50 μL of diluted cell lysate were transferred to a fresh 96 well culture plate. The assay was initiated with the addition of 50 μL of substrate per well. Color development was allowed to proceed for 10-15 minutes. The assay was terminated with the addition of 50 μL of Stop Solution (included in the Promega Kit). Optical densities were determined spectrophotometrically at 490 nm in a 96 well plate reader (SpectraMax 250, Molecular Devices, Sunnyvale, Calif., U.S.A.).

Test compounds were dissolved in 100% DMSO to prepare 10 mM stocks. Stocks were further diluted 1:250 in growth medium to yield working stocks of 40 μM test compound in 0.4% DMSO. Test compounds were serially diluted in growth medium containing 0.4% DMSO to maintain constant DMSO concentrations for all wells. 50 μL of fresh growth medium and 50 μL of diluted test compound were added to each culture well to give a final volume of 200 μL. The cells with and without individual test compounds were incubated for 72 hours at which time LDH activity was measured to yield T₇₂ₕ values. Optionally, the IC₅₀ values can be determined with a least squares analysis program using compound concentration versus percent inhibition.

% Inhibition=[1−(T_{72h test}−T₀ₕ)/(T_{72h ctrl}−T₀ₕ)]×100

where

T_{72h test}=LDH activity at 72 hours in the presence of test compound

T_{72h ctrl}=LDH activity at 72 hours in the absence of test compound

T₀ₕ=LDH activity at Time Zero

Representative results are shown in Tables 2A and 2B below:

TABLE 2A

| | % inhibition at a concentration |
|---|---|
| Example No. | of 10 μM |
| 5 | 87 |
| 7 | 85 |
| 8 | 84 |

TABLE 2B

| | % inhibition at a concentration |
|---|---|
| Example No. | of 2 μM |
| 12 | 73 |
| 23 | 90 |
| 46 | 90 |
| 49 | 91 |
| 50 | 77 |
| 52 | 81 |
| 56 | 64 |
| 57 | 89 |
| 61 | 90 |
| 66 | 88 |
| 68 | 43 |
| 71 | 72 |
| 72 | 81 |
| 76 | 63 |
| 81 | 89 |
| 87 | 95 |
| 88 | 40 |

In vivo Tumor Growth Inhibition Assay: MDA-MB-231 Tumor Xenograft Model

Inhibition of tumor growth in vivo is readily determined via the following assay:

MDA-MB-231 cells are cultured as described above. The cells are harvested by trypsinization, washed, counted, adjusted to 2.5×10⁷ cells/mL with ice cold phosphate-buffered saline (PBS), and subsequently stored on ice until transplantation. Xenograft experiments are conducted using eight-to-ten week-old female athymic mice with an average body mass of about 20-25 g. Approximately 5×10⁶ cells in a total volume of 0.2 mL PBS are injected subcutaneously in the flank region. Thereafter the mice are randomized and divided into several groups that reflect different dosages or schedules, respectively (n=10 mice/group). The test compounds are administered starting at day 1 at different dosages (e.g. 10, 20 and 40 mg/kg) and different schedules (e.g. Q1Dx15, Q2Dx7, Q3Dx5). Test compounds are formulated for oral administration in a vehicle for oral administration composed of polyethylene glycol-400, Cremophor®, ethanol and 0.9% saline (40:5:5:50). Tumor measurements are performed twice per week. Tumor weights are calculated using the formula (a×w²)/2. Animals are sacrificed on day 15 after transplantation and plasma was harvested for pharmacokinetic analyses.

In vivo Tumor Growth Inhibition Assay: MX-1 Tumor Xenograft Model

An MX-1 breast tumor xenograft model is maintained by serial passage in NCr nu/nu female mice (Taconic Farms, Germantown, N.Y.). Tumors are aseptically harvested from mice when they weigh approximately 1 g. The envelope and any non-viable areas are dissected and the viable tissue is cut into 3×3×3 mm cubes. These fragments are implanted in the axilary region of the flank of recipient mice using a trochar.

Treatment in anti-tumor efficacy studies is intiated when all mice have tumors ranging in size from 75-125 mg. There are typically 10 mice in each experimental group. Each experiment contains an untreated control group to monitor tumor growth kinetics, a vehicle-treated control group, and a positive agent control group to assess the response of the model in each experiment to an agent with an expected degree of anti-tumor efficacy. Lack of conformance of any of the controls to the historical ranges for the model constitutes a reason to nullify the study. The test compounds were administered starting at different dosages (e.g. 75 and 150 mg/kg) and different schedules (e.g. q1d×10, bid×10). Test compounds are formulated for oral administration once per day in a vehicle composed of 51% PEG400/12% ethanol/12% Cremophor EL/0.1 N HCl. Tumor size is recorded in whole mm as measured in two perpendicular dimensions. Animal body weights are recorded in tenths of grams. Both measurements are collected two to three times per week. Animals are sacrificed on day 10 after the last dose and last measurements.

Tumor weights are calculated using the equation (l×w²)/2, where l and w refer to the larger and smaller dimensions collected at each measurement. Efficacy is measured as the percent suppression of tumor growth expressed as %ΔT/ΔC, where ΔT and ΔC represent the change in the size of the average tumor in the treated and control groups, respectively, over the treatment period. Significance is evaluated using a Student's t-test with a p<0.05.

Abbreviations Used in this Specification

| | |
|---|---|
| BSA | bovine serum albumin |
| calc. | calculated |
| Cremophor ® | non-ionic emulsifyer from BASF |
| DBU | 1,8-diazabicyclo[5.4.0]undec-7-ene |
| DMEM | Dulbecco's Modified Eagle Medium, Life |
| | Technologies, Gaithersburg, MD, U.S.A. |
| DMF | N,N-dimethyl formamide |
| DMSO | dimethyl sulphoxide |
| EDTA | ethylene diamine tetraacetate |
| FBS | fetal bovine serum |
| HEPES | N-(2-hydroxyethyl)-piperazine-N′-(2- |
| | ethane sulphonic acid) |
| HPLC | high pressure liquid chromatography |
| HPLC-ES | high pressure liquid chromatography - |
| | coupled electrospray mass spectroscopy |
| LC-MS | liquid chromatography - coupled mass |
| | spectroscopy |
| LC RT | liquid chromatography retention time |
| LDH | lactate dehydrogenase |
| mp. | melting point |
| NMR | nuclear resonance spectroscopy |
| PBS | phosphate-buffered saline |
| TFA | trifluoroacetic acid |
| tlc | thin layer chromatography |
| Tris/HCl | tris(hydroxymethyl)-aminomethane |
| | hydrochloride |
| Triton ® X-100 | tert.-octylphenoxypolyethoxyethanol |

### EXAMPLES

The yield percentages of the following examples refer to the starting component which was used in the lowest molar amount.

A. LC-MS/HPLC Methods:

| Method A: | | |
|---|---|---|
| MS equipment: | Micromass Quattro LCZ | |
| | ionisation mode: | ESI positive/negative |
| HPLC equipment: | HP 1100 | |
| | UV detection: | 208-400 nm |
| | temperature: | 40° C. |
| Column: | ™ Symmetry C 18 | |
| | 50 mm × 2.1 mm | 3.5 μm |
| Supplier: | Waters | |

| | Time | | | Flow |
|---|---|---|---|---|
| Gradient: | [min.] | A: % | B: % | [mL/min.] |
| | 0.00 | 10.0 | 90.0 | 0.50 |
| | 4.00 | 90.0 | 10.0 | 0.50 |
| | 6.00 | 90.0 | 10.0 | 0.50 |
| | 6.10 | 10.0 | 90.0 | 1.00 |
| | 7.50 | 10.0 | 90.0 | 0.50 |

A: 0.1% strength solution of formic acid in acetonitrile
B: 0.1% strength aqueous formic acid

| Method B: | |
|---|---|
| Column: | ™ Kromasil C 18 |
| | 60 mm × 2.0 mm |

| | Time | | | Flow |
|---|---|---|---|---|
| Gradient: | [min.] | A: % | B: % | [mL/min.] |
| | 0.00 | 90.0 | 10.0 | 0.75 |
| | 0.50 | 90.0 | 10.0 | 0.75 |
| | 4.50 | 10.0 | 90.0 | 0.75 |
| | 6.50 | 10.0 | 90.0 | 0.75 |
| | 7.50 | 90.0 | 10.0 | 0.75 |

A: 0.001% strength aqueous H₃PO₄
B: acetonitrile

| Method C: | | | | |
|---|---|---|---|---|
| MS equipment: | | Micromass TOF-MUX-Interface 4-fold | | |
| | | parallel injection | | |
| | | ionisation mode: | | ESI positive |
| HPLC equipment: | | Waters 600 | | |
| | | UV detection: | | 210 nm |
| | | temperature: | | 40° C. |
| Column: | | Symmetry C 18 | | |
| | | 50 mm × 2.1 mm | | 3.5 μm |
| Supplier: | | Waters | | |
| | Time | | | Flow |
| Gradient: | [min.] | A: % | B: % | [mL/min.] |
| | 0.00 | 10.0 | 90.0 | 0.75 |
| | 0.50 | 10.0 | 90.0 | 0.75 |
| | 4.00 | 90.0 | 10.0 | 0.75 |
| | 5.50 | 90.0 | 10.0 | 0.75 |
| | 5.60 | 10.0 | 90.0 | 1.25 |
| | 6.50 | 10.0 | 90.0 | 0.75 |

A: 0.1% strength solution of formic acid in acetonitrile
B: 0.1% strength aqueous formic acid

| Method D: | | |
|---|---|---|
| MS equipment: | Micromass Platform LCZ | |
| | ionisation mode: | ESI positive/negative |
| HPLC equipment: | HP 1100 | |
| | UV detection: | 208-400 nm |
| | temperature: | 40° C. |
| Column: | Symmetry C 18 | |
| | 50 mm × 2.1 mm | 3.5 μm |
| Supplier: | Waters | |

| | Time | | | Flow |
|---|---|---|---|---|
| Gradient: | [min.] | A: % | B: % | [mL/min.] |
| | 0.00 | 10.0 | 90.0 | 0.50 |
| | 4.00 | 90.0 | 10.0 | 0.50 |
| | 6.00 | 90.0 | 10.0 | 0.50 |
| | 6.10 | 10.0 | 90.0 | 1.00 |
| | 7.50 | 10.0 | 90.0 | 0.50 |

A: 0.1% strength solution of formic acid in acetonitrile
B: 0.1% strength aqueous formic acid

| Method E: | |
|---|---|
| Column: | Kromasil C 18 |
| | 60 mm × 2.0 mm |

| | Time | | | Flow |
|---|---|---|---|---|
| Gradient: | [min.] | A: % | B: % | [mL/min.] |
| | 0.00 | 98.0 | 2.0 | 0.75 |
| | 4.50 | 10.0 | 90.0 | 0.75 |
| | 6.50 | 10.0 | 90.0 | 0.75 |
| | 6.70 | 98.0 | 2.0 | 0.75 |
| | 7.50 | 98.0 | 2.0 | 0.75 |

A: 0.5% strength aqueous HClO₄
B: acetonitrile

| Method F: | | |
|---|---|---|
| HPLC Equipment: | Gilson 215 | |
| | UV Detection: | 220 and 254 nM |
| | Temperature: | 25° C. |
| Column: | ™ YMC-Pack Pro C18 | |
| | 150 mm × 20 mm | 5 μm |
| Supplier: | Waters | |

| | Time | | | Flow |
|---|---|---|---|---|
| Gradient: | [min.] | A: % | B: % | [mL/min] |
| | 0:10 | 10.0 | 90.0 | 25.00 |
| | 11:00 | 100.0 | 0.0 | 25.00 |
| | 14.00 | 100.0 | 0.0 | 25.00 |
| | 15:02 | 10.0 | 90.0 | 25.00 |
| | 15:30 | 10.0 | 90.0 | 25.00 |

A: 0.1% strength solution of TFA in acetonitrile
B: 0.1% strength aqueous TFA

Method G:
(LCMS/ES Method)

| | | |
|---|---|---|
| MS equipment: | Finnigan LCQ Ion | ESI |
| | Trap Mass Spectrometer | |
| | ionisation mode: | |
| HPLC equipment: | HP 1100 | 254 nm |
| | UV detection: | |
| Column: | YMC pro C-18 | 120 Å |
| | 23 mm × 2 mm | |
| Supplier: | YMC | |

| | Time | | | Flow |
|---|---|---|---|---|
| Gradient: | [min.] | A: % | B: % | [mL/min.] |
| | 0.50 | 90.0 | 10.0 | 1.0 |
| | 3.50 | 5.0 | 95.0 | 1.0 |
| | 4.00 | 5.0 | 95.0 | 1.0 |
| | 4.00 | 90.0 | 10.0 | 1.0 |
| | 6.50 | 90.0 | 10.0 | 1.0 |

A: 0.02% strength solution of trifluoroacetic acid in 2% acetonitrile/98% water
B: 0.018% strength solution of trifluoroacetic acid in 98% acetonitrile/2% water

### B. Starting Materials

### Starting Material 1. Phenethyl Amines

The substituted 2-phenethyl amines are commercially available or can be prepared in analogy to anyone of the following procedures, e.g. starting from the corresponding benzaldehydes (see also Shepard et al. in J. Org. Chem. 17, 568 (1952) and in J. Am. Chem. Soc. 72, 4364 (1950)).

2-[3-(Trifluoromethoxy)-phenyl]-ethyl amine was obtained by hydrogenation of 3-[3-(trifluoromethoxy)-phenyl]-propane nitrile in analogy to the method described by Shepard et al. in J. Org. Chem. 17, 568 (1952).

### Intermediates 1. Amides

### 1.1. Ethyl 3-{[2-(3,4-dimethoxyphenyl)-ethyl]-amino}-3-oxopropanoate

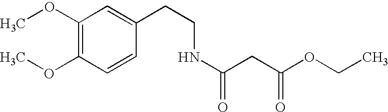

To a solution of 50.0 g (275.88 mmol) of 3,4-dimethoxyphenethylamine in 500 mL of dichloromethane was added 42.0 g (275.88 mmol) of 1,8-diazabicyclo[5.4.0]-undec-7-ene, followed by dropwise addition of 35.0 mL (41.62 g, 276.43 mmol) of ethyl malonyl chloride at a rate that kept the internal temperature below 30° C. The resultant clear yellow solution was stirred at room temperature under an argon atmosphere for 16 hours, at which time TLC analysis (silica gel 60, methanol/dichloromethane (5:95), UV detection) suggested complete reaction. The organics were washed with brine (3×1000 mL), dried over sodium sulfate and concentrated. The residue was dried under high vacuum at 30° C. for 24 hours to provide 80.55 g (272.75 mmol, 99%) of a yellow oil.

¹H-NMR (DMSO-d₆): δ=1.16 (t, J=7.0 Hz, 1.5H); 1.18 (t, J=7.0 Hz, 1.5H); 2.63 (t, J=7.7 Hz, 2H); 3.18 (s, 2H); 3.25 (m, 2H); 3.70 (s, 3H); 3.73 (s, 3H); 4.05 (q, J=7.0, 2H); 6.69 (dd, J=2.2 Hz, 8.4 Hz, 1H); 6.79 (d, J=2.2 Hz, 1H); 6.83 (d, J=8.4 Hz, 1H); 8.1 (bt, J=5.4 Hz, 1H).

MS (HPLC/ES; method G): m/z=296 (M+1).

TLC [methanol/dichloromethane (1:9)]: R_{f}=0.70.

The following Intermediates were obtained according to an analogous procedure:

1.2. Methyl 3-{[2-(3,4-dimethoxyphenyl)-ethyl]-amino}-3-oxopropanoate

1.3. Ethyl 3-{[2-(3-methoxy-4-ethoxyphenyl)-ethyl]-amino}-3-oxopropanoate

1.4. Ethyl 3-{[2-(3-methoxy-4-propoxyphenyl)-ethyl]-amino}-3-oxopropanoate

1.5. Methyl 3-{[2-(2-methoxy-3-methoxyphenyl)-ethyl]-amino}-3-oxopropanoate

1.6. Ethyl 3-{[2-(3-trifluoromethoxy-phenyl)-ethyl]-amino}-3-oxopropanoate

### Intermediates 2: (3,4-Dihydro-1(2H)-isoquinolinylidene)-ethanoates

### 2.1. Ethyl (6,7-dimethoxy-3,4-dihydro-1(2H)-isoquinolinylidene)-ethanoate

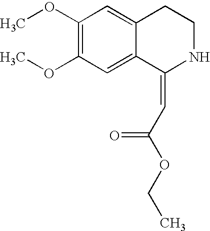

To a refluxing solution of methanesulfonic anhydride (648.83 g, 3.72 mol) in toluene (4 L) was added Intermediate 1.1, ethyl 2-{N-[2-(3,4-dimethoxyphenyl)-ethyl]-carbamoyl}-acetate, (1000 g, 3.39 mol) portionwise over 20 minutes. The reaction was stirred at reflux for 30 minutes at which point the heat was removed and the toluene was decanted. The resulting dark oil was then dissolved in water (3000 mL) and treated portionwise with solid potassium carbonate until a pH of about 8 was achieved. The organic material was extracted from the dark biphasic mixture using ethyl acetate (3000 mL). The combined organic extracts were washed with brine (3×2000 mL) and concentrated to 1/3 volume. The resultant dark oil was placed on a pad of silica gel 60 (400 cc) and eluted using ethyl acetate/hexane (1:1). The desired fractions were concentrated to a yellow oil which was seeded with a small amount of crystals of the title compound and placed in a refrigerator overnight. The yellow crystalline solid which formed was filtered, washed with ethyl acetate/hexane (1:1) (2×50 ml), and vacuum dried for 12 hours to give 533.26 g of the desired product. The filtrate was concentrated to a dark oil and seeded a second time. After 1 hour, the newly formed yellow solid was filtered, washed with ethyl acetate/hexane (1:1) (2×50 ml), and vacuum dried for 12 hours to provide 106.23 g of a second crop. The two batches of crystals were combined to provide the title compound (639.49 g, 68%).

¹H-NMR (DMSO-d₆): δ1.18 (t, J=7.0 Hz, 3H); 2.76 (t, J=6.5 Hz, 2); 3.36 (m, 2H); 3.78 (s, 6H); 4.02 (q, J=7.0 Hz, 2H); 5.05 (s, 1H); 6.87 (s, 1H); 7.15 (s, 1H); 8.95 (bs, 1H).

MS (HPLC/ES; method G): m/z=278 (M+1).

TLC [ethyl acetate/hexane (1:1)]: R_{f}=0.63

Instead of methanesulfonic anhydride also phosphorous pentoxide can be used according to this method.

### Intermediate 3. Ethyl (2E,Z)-(6-methoxy-3,4-dihydro-1(2H)-isoquinolinylidene)-ethanoate (3.1) and ethyl (2E,Z)-(8-methoxy-3,4-dihydro-1(2H)-isoquinolinylidene)-ethanoate (3.2)

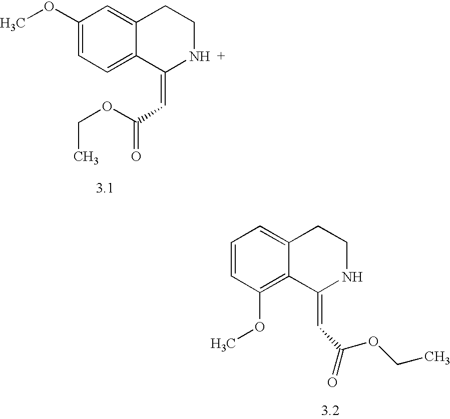

A solution of 44,10 g (170 mmol) of ethyl 3-{[2-(3-methoxyphenyl)-ethyl]-amino}-3-oxopropanoate (prepared in analogy to Intermediate 1 from 3-methoxy-phenylethyl amine and ethyl 3-chloro-3-oxopropanoate with 95,8% yield) in 432 mL of toluene was heated under reflux, and 179,31 g (1260 mmol) of phosphorus pentoxide were added to the boiling solution in 6 portions at 15-20 min. intervals (following the course of the reaction by tic using cyclohexane/ethyl acetate (1:1) as eluant). After cooling to room temperature, 1 L of water was added slowly with ice cooling, and then the resulting mixture was made alcaline by adding potassium carbonate. The aqueous phase was extracted 4 times with ether; the combined organic phases were dried over sodium sulphate, filtered and the solvent was evaporated. Compounds 3.1 and 3.2 were separated by silica gel chromatography: 20,5 g (48,89%) of compound 3.1 and 620 mg (1,51%) of compound 3.2 were obtained.

### Intermediate 4: 1H-benzimidazol-6-ylmethanol

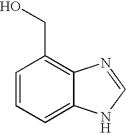

To a solution of 500 mg (3.08 mmol) of 1H-benzimidazole-6-carboxylic acid, 1.50 g (3.39 mmol) of benzotriazol-1-yloxy-tris(dimethylamino)-phosphonium hexafluoro-phosphonate and 0.810 mL (4.63 mmol) diisopropylethyl amine in 90 mL of anhydrous tetrahydrofuran was added 455 mg (12.0 mmol) of sodium borohydride. The mixture was stirred at room temperature for 3 hours, and the solvent was evaporated to give a residue, to which were added 100 mL of methanol and 25 mL of water. The resulting mixture was stirred for one hour to produce a clear solution. Removal of the solvent under reduced pressure gave an oily residue, which was subjected to filtration through a pad of silica gel using an ethyl acetate/hexane mixture (3:7) as eluent to afford 302 mg of crude 1H-benzimidazol-6-yl methanol (Intermediate 4).

### Intermediate 5: 1H-benzimidazol-6-yl carboxaldehyde

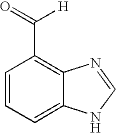

The crude Intermediate 4 residue was dissolved in 8 mL of acetonitrile and 1.00 g (500 mg/mmol substrate) of 4 Å molecular sieves. 358 mg (3.06 mmol) of N-methylmorpholine N-oxide monohydrate and 36 mg (0.10 mmol) of tetrapropylammonium perruthenate were added and the reaction was left to stir under an argon atmosphere at room temperature. After 45 minutes the reaction looked complete by TLC. The mixture was filtered through a pad of silica gel, eluting with ethyl acetate. The resulting solution was concentrated to afford 302 mg of a slightly pink colored solid crude product, 1H-benzimidazol-6-yl carboxaldehyde (Intermediate 5).

### Intermediate 6: Ethyl 2-(3-hydroxy-4-nitrophenyl)-8,9-dimethoxy-3-methyl-5,6-dihydropyrrolo[2,1-a]isoquinoline-1-carboxylate

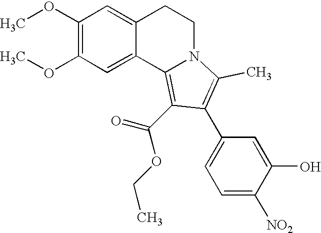

A mixture of 830 mg (2.99 mmol) of ethyl (2E)-(6,7-dimethoxy-3,4-dihydro-1(2H)-isoquinolinylidene)-ethanoate (Intermediate 2.1 above), 450 mg (5.98 mmol) of nitroethane, 0.71 mL (7.18 mmol) of piperidine, and 1.00 g (5.98 mmol) of 3-hydroxy-4-nitro-benzaldehyde in 15 mL of ethanol and 15 mL of isopropanol was heated at 70° C. overnight. The solution was allowed to cool to room temperature. Some slightly yellow solid precipitated out and was collected by filtration to give 1.10 g (81%) pure product.

MS (HPLC/ES): m/z=453.0 (M+1). HPLC RT: 3.54 min.

### Intermediate 7: N-benzyl indoline

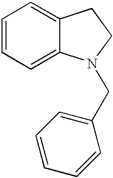

To a solution of 2.60 g (21.82 mmol) of indoline in 75 mL of tetrahydrofuran at −78° C. was added 15.00 mL (24.00 mmol) of 1.6 M n-butyllithium in hexane. After 15 minutes of stirring, 4.29 g (25.09 mmol) of benzyl bromide were added, and the reaction was allowed to warm to room temperature over 1 hour at which time no starting indoline remained by TLC analysis (silica gel 60, 100% ethyl acetate, UV detection). The reaction was quenched with 35 mL of water, and most of the tetrahydrofuran was removed by rotary evaporation. The residue was extracted with ethyl acetate (3×100 mL), and the combined organic phases were dried with sodium sulfate and concentrated to give 4.60 g of N-benzyl indoline (21.8 mmol, 99%) as a faintly orange solid.

### Intermediate 8: N-benzyl indol-5-yl carboxaldehyde

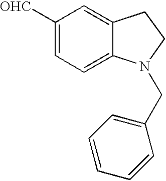

Intermediate 7 was taken up in 50 mL of dichloroethane and added to a pre-prepared solution of 3.04 mL (32.35 mmol) of phosphorous oxychloride and 2.04 mL (32.25 mmol) of dimethylformamide in 10 mL of dichloroethane maintained at 0° C. The reaction mixture was then heated to 50° C. for 4 hours and subsequently poured into 350 mL of precooled 15% aqueous sodium acetate solution. The mixture was left to stir for 1 hour at 0° C. and for 2 hours at room temperature and was then extracted with dichloromethane (3×100 mL). The combined organic phases were washed with saturated aqueous sodium bicarbonate solution and dried over magnesium sulfate. Concentration in vacuo gave 3.65 g (15.5 mmol, 72%) of the 5-carboxaldehyde derivative (Intermediate 8) as a yellow crystalline solid.

### Intermediate 9: 7-methoxyindole-3-carboxaldehyde

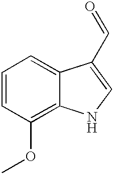

To a solution of phosphorous oxychloride (190 μL, 2.04 mmol) in DMF (2.5 mL) at 0° C. was added 7-methoxy indole (220 μL, 1.70 mmol). The reaction mixture was allowed to warm to room temperature and to stir for 2 hours. The solution was diluted with dichloromethane and made basic with aqueous 1N sodium hydroxide solution. The layers were separated and the organic layer was washed with aqueous 1N sodium hydroxide solution (2×10 mL). The organic layer was dried (MgSO₄), concentrated in vacuo, and the crude product was purified by eluting through a pad of silica with dichlormethane, to afford Intermediate 9 as light brown needles (284 mg, 95%).

### C. Preparation Examples

### Example 1

### Ethyl 2-(1H-indol-4-yl)-8,9-dimethoxy-3-methyl-5,6-dihydropyrrolo[2,1-a]isoquinoline-1-carboxylate

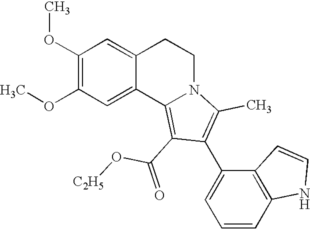

A mixture of 500 mg (1.8 mmol) of ethyl (6,7-dimethoxy-3,4-dihydro-1(2H)-isoquinolinylidene)-ethanoate (Intermediate 2.1), 523 mg (3.61 mmol) of indole-4-carbaldehyde, 281 mg (3.61 mmol) of nitroethane and 61.4 mg (0.72 mmol) of piperidine in 10 mL of ethanol/isopropanol (1:1) was stirred at 80° C. overnight. 40 mL of isopropanol were added, the mixture was cooled to 0° C., and the resulting precipitate was filtered off. The solid was washed with ethanol and dried in vacuo to give the title compound as a white solid. The title compound was readily recrystallized from ethyl acetate to furnish white needles.

Yield: (487 mg, 63%)

Melting point [° C.]: 246-247

The following compounds were prepared in analogy to the description of Example 1. All aldehydes are commercially available or are prepared in analogy to published procedures (Compendium of Organic Synthetic Methods, I. T. and S. Harrison; Wiley-Interscience, Inc., pages 132-177). If nitropropane or nitropentane is used instead of nitroethane, the corresponding ethyl 3-ethyl-5,6-dihydro-pyrrolo[2,1-a]isoquinolines or ethyl 3-butyl-5,6-dihydro-pyrrolo[2,1-a]isoquinolines are obtained.

| Ex. | Structure | Analytical data |
|---|---|---|
| 2 | 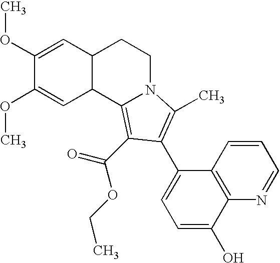 | ¹H NMR (300 MHz, DMSO-d₆): δ=0.34 (t, J=7.2 Hz, 3H), 1.98 (s, 3H), 3.01 (t, J=6.2 Hz, 2H), 3.56-3.68 (m, 2H), 3.73 (s, 3H), 3.81 (s, 3H), 4.00 (t, J=6.4 Hz, 2H), 6.97 (s, 1H), 7.09 (d, J=7.7 Hz, 1H), 7.21 (d, J=7.9 Hz, 1H), 7.47 (dd, J=8.5 Hz, J=4.2 Hz, 1H), 7.89 (s, 1H), 7.95 (dd, J=8.5 Hz, J=1.5 Hz, 1H), 8.82 (dd, J=4.0 Hz, J=1.5 Hz, |
| # 1H), 9.56 (bs, 1H) MS: 459.3 (M + H) HPLC retention time [min.]: 3.63 (method C) | | |
| 3 | 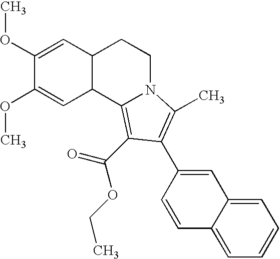 | MS: 443.0 (M + H) HPLC retention time [min.]: 3.65 (method B) |
| 4 | 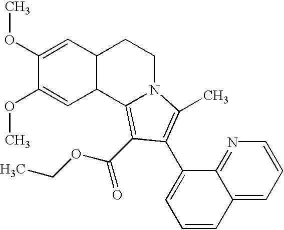 | ¹H NMR (200 MHz, DMSO-d₆): δ=0.19 (t, J=7.1 Hz, 3H), 2.10 (s, 3H), 3.01(t, J=6.2 Hz, 2 H), 3.73 (s, 3H), 3.81 (s, 3H), 4.01 (t, J=6.2 Hz, 2H), 6.97 (s, 1H), 7.48 (q, J=4.1 Hz, 1H), 7.52-7.67 (m, 2H), 7.90 (dd, J=7.6 Hz, J=2.0 Hz, 1H), 8.01 (s, 1H), 8.35 (dd, J=8.2 Hz, J=1.7 Hz, 1H), 8.75 (dd, J=4.2 Hz, J=1.8 Hz, 1H) MS: |
| # 443.1 (M + H) HPLC retention time [min.]: 5.93 (method B) | | |
| 5 | 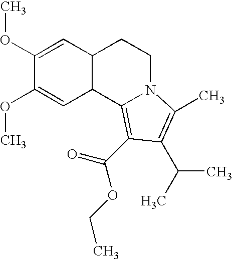 | MS: 358.3 (M + H) HPLC retention time [min.]: 4.8 (method D) |
| 6 | 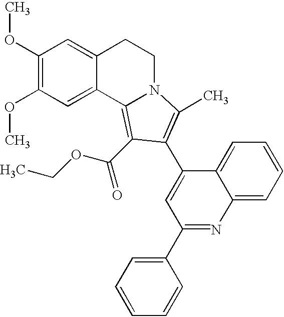 | ¹H NMR (200 MHz, DMSO-d₆): δ =0.26 (t, J=7.1 Hz, 3H), 2.09 (s, 3H), 3.04 (bs, 2H), 3.47-3.69 (m, 2H), 3.75 (s, 3H), 3.82 (s, 3H), 4.06 (t, J=6.2 Hz, 2H), 7.01 (s, 1H), 7.39-7.61 (m, 4H), 7.64-7.81 (m, 2H), 7.93 (s, 1H), 8.04 (s, 1H), 8.11 (d, J=8.2 Hz, 1H), 8.25-8.36 (m, 2H) MS: 519.2 (M + H) HPLC retention time [min.]: 4.97 (method B) |
| 7 | 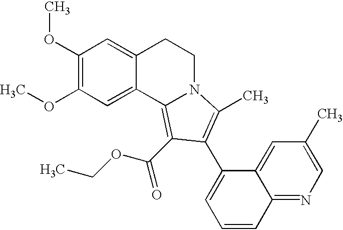 | Melting point [° C.]: 120-122 |
| 8 | 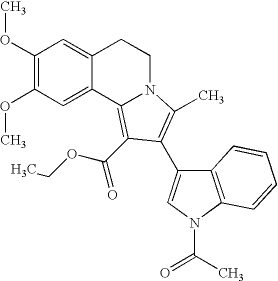 | MS: 473.1 (M + H), 495.1 (M + Na) HPLC retention time [min.]: 4.96 (method E) |
| 9 | 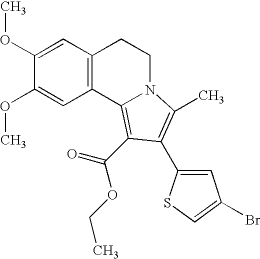 | MS: 478.1 (M + H) HPLC retention time [min.]: 5.01 (method A) |
| 10 | 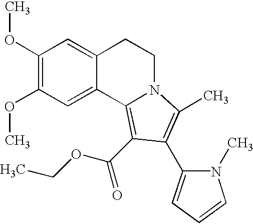 | ¹H NMR (200 MHz, CDCl₃): d=0.99 (t, J=7.0 Hz, 3H), 2.13 (s, 3H), 2.91-3.01 (m, 2H), 3.41 (s, 3H), 3.83-4.14 (m, 4H), 3.90 (s, 3H), 3.92 (s, 3H), 5.97 (d, J=3.4 Hz, 1H), 6.14 (d, J=3.4 Hz, 1H), 6.58-6.76 (m, 1H), 6.71 (s, 1H), 8.08 (s, 1H) |
| 11 | 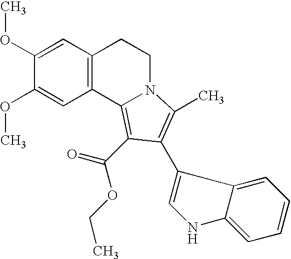 | MS: 431.3 (M + H) HPLC retention time [min.]: 4.41 (method D) |
| 12 | 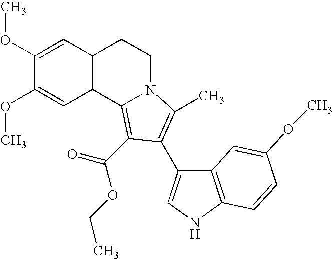 | ¹H NMR (200 MHz, DMSO-d₆): δ =0.67 (t, J=7.2 Hz, 3H), 2.15 (s, 3H), 2.91-3.05 (m, 2H), 3.61-3.90 (m, 2H), 3.67 (s, 3H), 3.72 (s, 3H), 3.79 (s, 3H), 3.91-4.06 (m, 2H), 6.62-6.75 (m, 2H), 6.95 (s, 1H), 7.15 (d, J=2.4 Hz, 1H), 7.26 (d, J=9.4 Hz, 1H), 7.61 (s, 1H), 10.86 (s, 1H) MS: 461.3 (M + H) HPLC retention time [min.]: 4.2 (method A) |
| 13 | 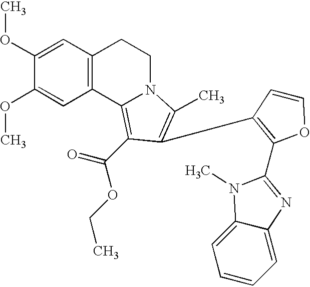 | MS: 512.3 (M + H) HPLC retention time [min.]: 3.3 (method A) |
| 14 | 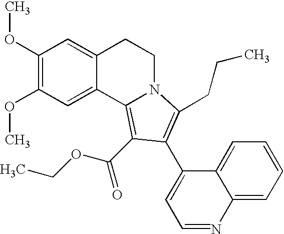 | MS: 471.3 (M + H) HPLC retention time [min.]: 3.44 (method A) |
| 15 | 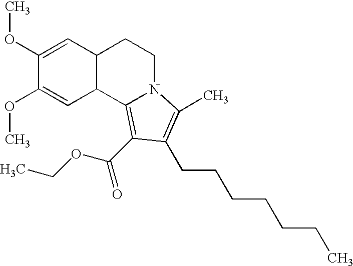 | ¹H NMR (300 MHz, CDCl₃): δ = 0.82-0.92 (m, 3H), 1.19-1.55 (m, 13H), 2.16 (s, 3H), 2.62 (t, J=7.7 Hz, 2H), 2.92 (t, J=6.4 Hz, 2H), 3.83 (t, J=6.2 Hz, 2H), 3.88 (s, 3H), 3.89 (s, 3H), 4.31 (q, J=7.0 Hz, 2H), 6.68 (s, 1H), 7.80 (s, 1H) MS: 414.4 (M + H) HPLC retention time [min.]: 6.42 (method B) |
| 16 | 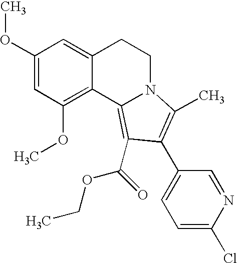 | Melting point [° C.]: 187-188 |
| 17 | 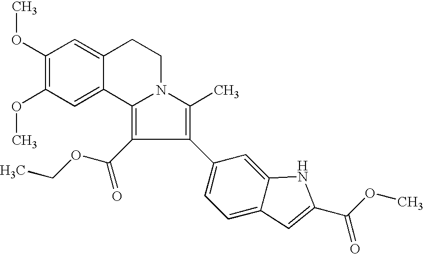 | Melting point [° C.]: 238-240 |
| 18 | 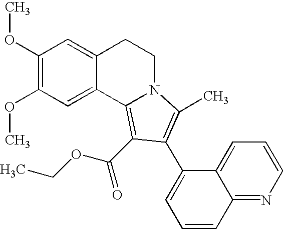 | ¹H NMR (200 MHz, DMSO-d₆): δ =0.24 (t, J=7.2 Hz, 3H), 2.00 (s, 3H), 3.02 (t, J=6.2 Hz, 2H), 3.47-3.66 (m, 2H), 3.73 (s, 3H), 3.81 (s, 3H), 4.02 (t, J=6.4 Hz, 2H), 6.98 (s, 1H), 7.34-7.50 (m, 2H), 7.69-7.82 (m, 1H), 7.92-8.05 (m, 2H), 7.96 (s, 1H), 8.81-8.91 (m, 1H) MS: 443.1 (M + H) HPLC retention time [min.]: 4.3 (method B) |
| 19 | 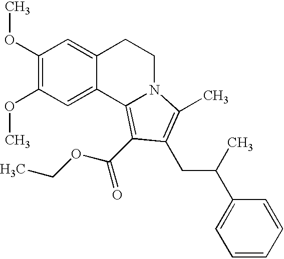 | ¹H NMR (300 MHz, CDCl₃): δ = 1.27 (d, J=6.4 Hz, 3H), 1.34 (t, J=7.0 Hz, 3H), 1.80 (s, 3H), 2.73-3.01 (m, 5H), 3.76 (t, J=6.6 Hz, 2H), 3.89 (s, 3H), 3.91 (s, 3H), 4.23-4.43 (m, 2H), 6.68 (s, 1H), 7.09-7.30 (m, 5H), 7.80 (s, 1H) MS: 434 (M + H) HPLC retention time [min.]: 5.63 (method B) |
| 20 | 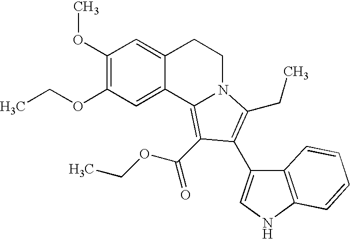 | HPLC retention time [min.]: 4.73 (method D) |
| 21 | 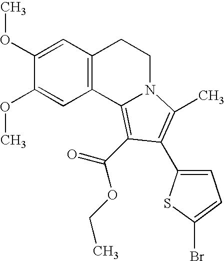 | MS: 478.1 (M + H) HPLC retention time [min.]: 5.09 (method A) |
| 22 | 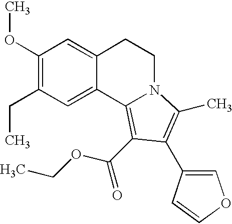 | ¹H NMR (200 MHz, CDCl₃): δ = 1.14 (t, J=7.2 Hz, 3H), 2.21 (s, 3H), 2.97 (t, J=6.6 Hz. 2H), 3.82-3.97 (m, 8H), 4.18 (q, J=7.2 Hz, 2H), 6.43 (s, 1H), 6.71 (s, 1H), 7.39 (s, 1H), 7.44 (s, 1H), 7.81 (s, 1H) MS: 382.1 (M + H), 399.0 (M + NH₄) HPLC retention time [min.]: 4.77 (method B) |
| 23 | 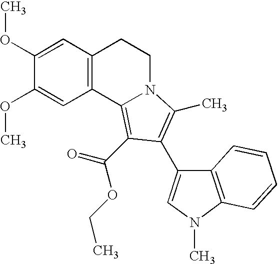 | MS: 445.3 (M + H) HPLC retention time [min.]: 4.78 (method D) |
| 24 | 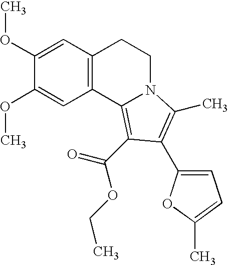 | MS: 396.3 (M + H) HPLC retention time [min.]: 4.69 (method D) |
| 25 | 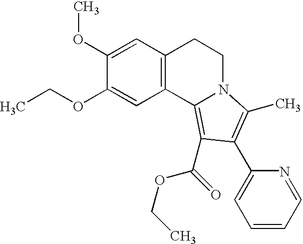 | Melting point [° C.]: 133-134 |
| 26 | 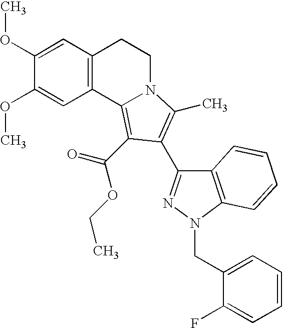 | MS: 540.4 (M + H) HPLC retention time [min.]: 4.93 (method A) |
| 27 | 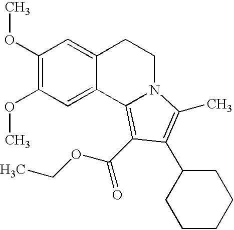 | ¹H NMR (300 MHz, CDCl₃): δ = 1.14-1.87 (m, 13H), 2.23 (s, 3H), 2.80 (tt, J=11.7 Hz, J=3.8 Hz, 1H), 2.92 (t, J=6.6 Hz, 2H), 3.82 (t, J=6.8 Hz, 2H), 3.85 (s, 3H), 3.87 (s, 3H), 4.33 (q, J=7.2 Hz, 2H), 6.67 (s, 1H), 7.36 (s, 3H) MS: 398.0 (M + H) HPLC retention time [min.]: 5.64 (method B) |
| 28 | 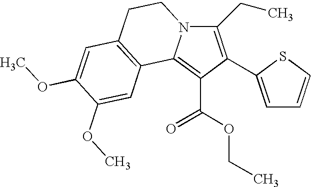 | |
| 29 | 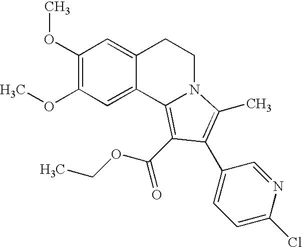 | Melting point [° C.]: <250 |
| 30 | 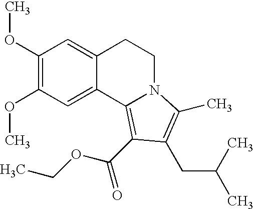 | ¹H NMR (300 MHz, CDCl₃): δ = 0.89 (d, J=6.6 Hz, 6H), 1.35 (t, J=7.2 Hz, 3H), 1.79 (sept, J=6.8 Hz, 1H), 2.15 (s, 3H), 2.52 (d, J=7.0 Hz, 2H), 2.92 (t, J=6.4 Hz, 2H), 3.84 (t, J=6.8 Hz, 2H), 3.88 (s, 3H), 3.89 (s, 3H), 4.31 (q, J=7.0 Hz, 2H), 6.68 (s, 1H), 7.78 (s, 1H) MS: 372.0 (M + H) HPLC retention time [min.]: 5.45 (method B) |
| 31 | 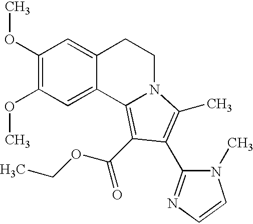 | ¹H NMR (300 MHz, DMSO-d₆): δ =0.88 (t, J=7.2 Hz, 3H), 2.05 (s, 3H), 2.97 (t, J=6.4 Hz, 2H), 3.39 (s, 3H), 3.75 (s, 3H), 3.80 (s, 3H), 3.84-4.05 (m, 4H), 6.89 (d, J=1.0 Hz, 1H), 6.95 (s, 1H), 7.16 (d, J=1.1 Hz, 1H), 8.00 (s, 1H) MS: 396.1 (M + H) HPLC retention time [min.]: 3.82 (method E) |
| 32 | 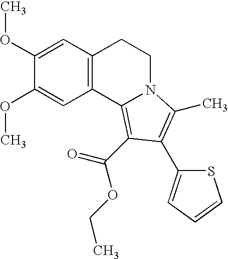 | MS: 398.2 (M + H) HPLC retention time [min.]: 4.77 (method D) |
| 33 | 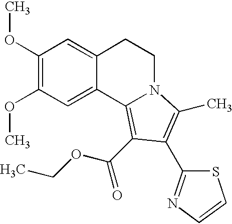 | ¹H NMR (200 MHz, DMSO-d₆): δ =1.00 (t, J=7.1 Hz, 3H), 2.34 (s, 3H), 2.97 (t, J=6.3 Hz, 2H), 3.73 (s, 3H), 3.79 (s, 3H), 3.98 (t, J=6.4 Hz, 2H), 4.04 (q, J=7.1 Hz, 2H), 6.97 (s, 1H), 7.58 (s, 1H), 7.26 (d, J=3.3 Hz, 1H), 7.82 (d, J=3.3 Hz, 1H) MS: 399.2 (M + H) HPLC retention time [min.]: 4.19 (method B) |
| 34 | 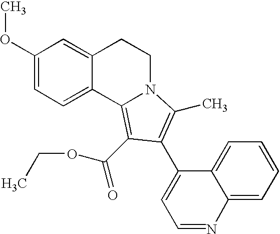 | Melting point [° C.]: 149-150 |
| 35 | 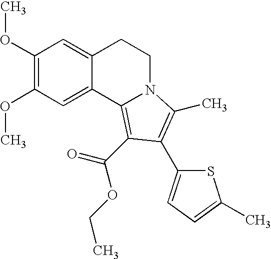 | MS: 412.3 (M + H) HPLC retention time [min.]: 4.96 (method D) |
| 36 | 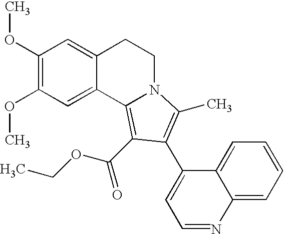 | ¹H NMR (300 MHz, CDCl₃): δ = 0.31 (t, J=7.2 Hz, 3H), 2.07 (s, 3H), 3.06 (t, J=6.6 Hz, 2H), 3.50-3.77 (m, 2H), 3.93 (s, 3H), 3.94 (s, 3H), 4.01 (t, J=6.2 Hz, 2H), 6.75 (s, 1H), 7.22-7.30 (m, 1H), 7.45 (dt, J=6.8 Hz, J=1.0 Hz, 1H), 7.67 (dt, J=7.0 Hz, J=1.3 Hz, 1H), 7.81 (d, J=8.3 Hz, 1H), 8.14 (d, J=8.3 Hz, 1H), 8.24 (s, 1H), 8.91 (d, J=4.5 Hz, |
| # 1H) MS: 443.3 (M + H) HPLC retention time [min.]: 3.54 (method B) | | |
| 37 | 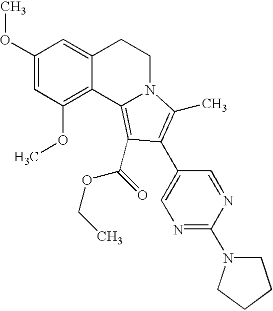 | Melting point [° C.]: 133-134 |
| 38 | 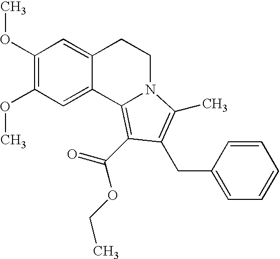 | MS: 406.3 (M + H) HPLC retention time [min.]: 4.93 (method D) |
| 39 | 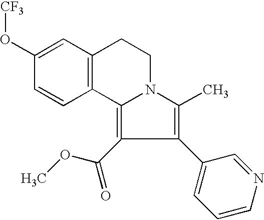 | Melting point [° C.]: 217-218 |
| 40 | 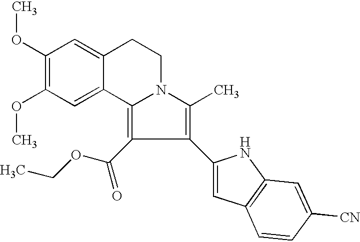 | Melting point [° C.]: 230 |
| 41 | 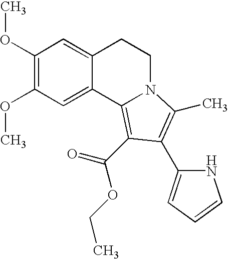 | MS: 381.2 (M + H) HPLC retention time [min.]: 4.69 (method E) |
| 42 | 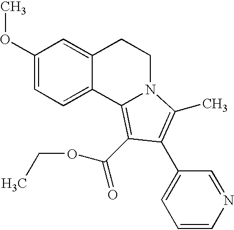 | Melting point [° C.]: 134-135 |
| 43 | 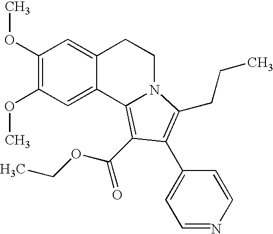 | MS: 421.2 (M + H) HPLC retention time [min.]: 2.964 (method A) |
| 44 | 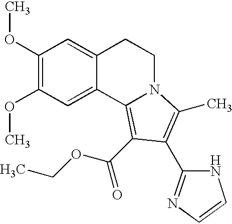 | MS: 382.3 (M + H) HPLC retention time [min.]: 3.68 (method E) |
| 45 | 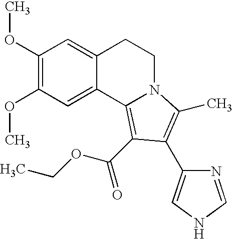 | MS: 382.2 (M + H) HPLC retention time [min.]: 3.77 (method E) |

### Example 46

### Ethyl-2-(1H-7-methoxy-indol-3-yl)-8,9-dimethoxy-3-methyl-5,6-dihydropyrrolo[2,1-a]isoquinoline-1-carboxylate

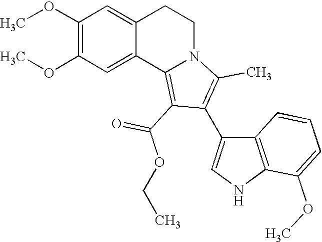

A mixture of ethyl (6,7-dimethoxy-3,4-dihydro-1(2H)-isoquinolinylidene)-ethanoate (Intermediate 2.1) (132 mg, 0.48 mmol), 7-methoxy indole (Intermediate 9) (125 mg, 0.71 mmol), nitroethane (54 mg, 0.71 mmol), and piperidine (12 mg, 0.14 mmol) in ethanol (10 mL) was stirred at 80° C. for 48 hours. The solvent was removed in vacuo, and the crude product was titurated with diethyl ether and cooled to 0° C. The resulting solid was filtered, washed with diethyl ether and dried in vacuo to give the title compound as a light yellow solid (26 mg, 12%).

MP ° C.: 246-247. HPLC RT (Method G): 2.94 min

¹H NMR (CD₃CN, 400 MHz) δ9.32 (bs, 1 H), 7.67 (s, 1 H), 7.13 (m, 1 H), 6.93 (m, 3 H), 6.69 (dd, J=7.4, 1.0 Hz, 1 H), 3.99 (m, 5 H), 3.93 (q, J=7.1 Hz, 2 H), 3.87 (s, 3 H), 3.81 (s, 3 H), 3.03 (t, J=6.4 Hz, 2 H), 2.18 (s, 3 H), 0.82 (t, J=7.0 Hz, 3 H)

The compounds of the following examples were prepared using the same method as that employed for the synthesis of Example 46.

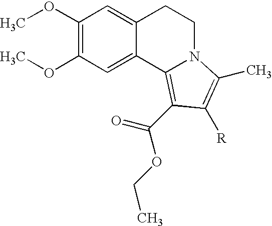

| | | LCMS | LC RT |
|---|---|---|---|
| | | m/z | Method G |
| Example | R | (M + 1) | (min) |
| 47 | 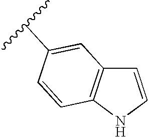 | 431.1 | 3.27 |
| 48 | 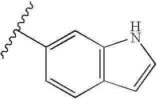 | 431.1 | 3.31 |
| 49 | 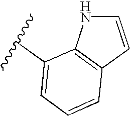 | 430.9 | 3.55 |
| 50 | 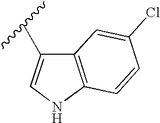 | 465.4 | 3.59 |
| 51 | 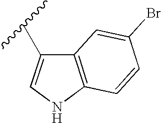 | 509.1 | 3.49 |
| 52 | 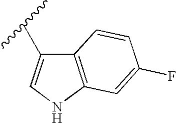 | 449.5 | 3.40 |
| 53 | 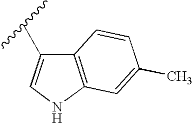 | 445.2 | 3.37 |
| 54 | 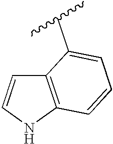 | 431.1 | 3.16 |
| 55 | 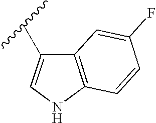 | 448.6 | 3.08 |
| 56 | 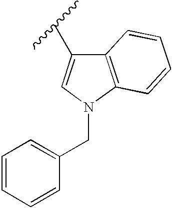 | 521.6 | 3.70 |

### Example 57

### Ethyl 2-(1-ethyl-1H-indol-4-yl)-8,9-dimethoxy-3-methyl-5,6-dihydropyrrolo[2,1-a]-isoquinoline-1-carboxylate

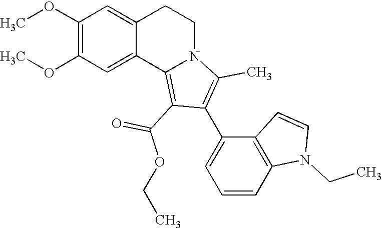

To a solution of 126 mg (0.29 mmol) of ethyl 2-(1H-indol-4-yl)-8,9-dimethoxy-3-methyl-5,6-dihydropyrrolo[2,1-a]isoquinoline-1-carboxylate (Example 46 above) and 5.4 mg (0.01 mmol) of tetrabutylammonium iodide in 2 mL of methylene chloride were added 90.25 mg (80 μL, 0.59 mmol) of diethyl sulfate and 1 mL of a 50% aqueous sodium hydroxide solution. The reaction mixture was allowed to stir for 15 hours at room temperature, at which time TLC analysis (silica gel 60, ethyl acetate/hexanes (2:3), UV detection) suggested complete reaction. The reaction mixture was diluted with 15 mL of dichloromethane and 15 mL of 1 N phospate buffer. The organic layer was washed with brine, dried over sodium sulfate, filtered and concentrated. The crude product was titurated with ether to afford the product as an off-white solid 1.75 g (4.07 mmol, 79%).

MS (HPLC/ES): m/z=459.0 (M+1). HPLC RT (Method G): 3.72.

The compounds of the following examples were prepared using the same method as that employed for the synthesis of Example 57:

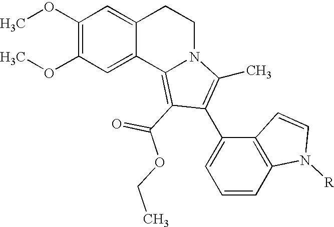

| | | LCMS | LC RT |
|---|---|---|---|
| | | m/z | Method G |
| Example | R | (M + 1) | (min) |
| 58 | 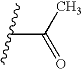 | 473.0 | 3.45 |
| 59 | 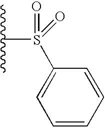 | 571.0 | 3.77 |
| 60 | 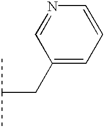 | 521.9 | 2.63 |
| 61 | 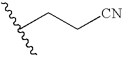 | 484.6 | 3.23 |
| 62 | 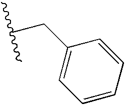 | 521.2 | 3.84 |
| 63 | 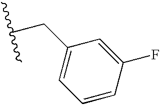 | 539.2 | 3.90 |
| 64 | 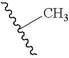 | 503.2 | 3.58 |
| 65 | 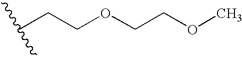 | 533.2 | 3.36 |

The compounds of the following examples were prepared using the same method as that employed for the synthesis of Example 57:

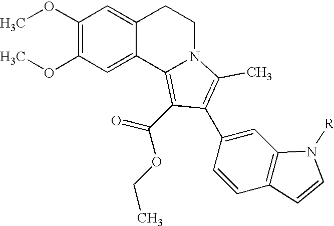

| | | LCMS | LC RT |
|---|---|---|---|
| | | m/z | Method G |
| Example | R | (M + 1) | (min) |
| 66 | 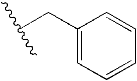 | 520.2 | 3.94 |
| 67 | 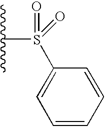 | 570.9 | 3.81 |

### Example 68

### Ethyl 2-[3-(cyclopropylcarbonyl)-1H-indol-7-yl]-8,9-dimethoxy-3-methyl-5,6-dihydropyrrolo[2,1-a]isoquinoline-1-carboxylate

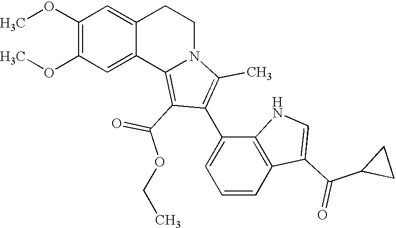

97 mg (0.37 mmol) of tin (II) chloride were added to a solution of 80 mg (0.19 mmol) of ethyl 2-(1H-indol-7-yl)-8,9-dimethoxy-3-methyl-5,6-dihydropyrrolo-[2,1-a]isoquinoline-1-carboxylate (prepared as described for Example 50 above) and 39 mg (0.37 mmol) of cyclopropanecarbonyl chloride in 1 mL of anhydrous toluene at 0° C. under an argon atmosphere. After being stirred for one hour at 0° C., the mixture was allowed to warm to room temperature and was stirred for an additional hour. Subsequently, 5 mL of water and 5 mL of ethyl acetate were added, and the two phases were separated. The organic layer was washed with 5 mL of water, dried over magnesium sulfate, filtered and concentrated. The resulting crude product was washed with ether and purified by HPLC (method F). The HPLC solvent was evaporated. The resulting solid was dissolved in ethyl acetate, and the organic layer was washed with a saturated aqueous sodium carbonate solution, dried over magnesium sulfate, filtered and run through a short silica gel column eluting with ethyl acetate to afford 26.8 mg (29%) of the title compound.

MS (HPLC/ES): m/z=499.2 (M+1). HPLC RT (Method G): 3.96 min.

The compounds of the following examples were prepared using the same method as that employed for the synthesis of Example 68:

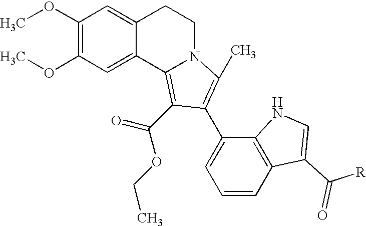

| | | | LC RT |
|---|---|---|---|
| | | HPLC/ES | Method G |
| Example | R | (M + 1) | (min) |
| 69 | 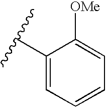 | 565.3 | 4.06 |
| 70 | 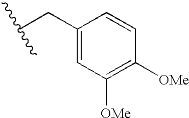 | 609.2 | 3.99 |
| 71 | 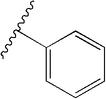 | 535.2 | 4.14 |

The compounds of the following examples can be prepared using the same method as synthesis of Example 68:

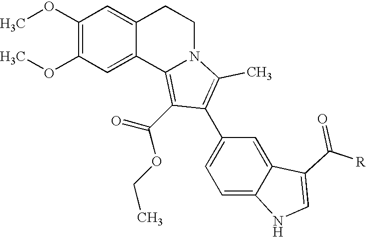

| | | | LC RT |
|---|---|---|---|
| | | HPLC/ES | Method G |
| Example | R | (M + 1) | (min) |
| 72 | 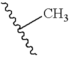 | 473.2 | 2.93 |
| 73 | 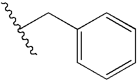 | 549.3 | 3.90 |
| 74 | 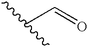 | 459 | 3.50 |

### Example 75

### Ethyl 2-(1H-benzimidazol-6-yl)-8,9-dimethoxy-3-methyl-5,6-dihydropyrrolo[2,1-a]isoquinoline-1-carboxylate

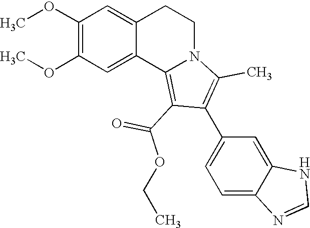

The Intermediate 5 residue was added to a solution of 286 mg (1.03 mmol) of ethyl (2E)-(6,7-dimethoxy-3,4-dihydro-1(2H)-isoquinolinylidene)-ethanoate (Intermediate 2.1 above), 155 mg (2.07 mmol) of nitroethane, and 211 mg (2.48 mmol) of piperidine in 10 mL of ethanol and 10 mL of isopropanol. The mixture was heated at 70° C. overnight. Removal of the solvents under reduced pressure gave an oily residue, which was purified by HPLC (Method F) to afford 4.5 mg (1.0%) of the title compound.

MS (HPLC/ES): m/z=432.3 (M+1). HPLC RT (Method G): 2.09 min.

### Example 76

### Ethyl 2-(1,3-benzoxazol-6-yl)-8,9-dimethoxy-3-methyl-5,6-dihydropyrrolo[2,1-a]isoquinoline-1-carboxylate

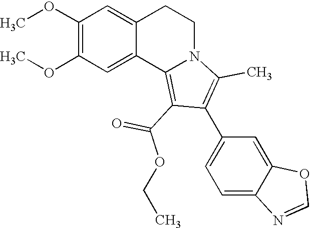

18.3 mg (0.08 mmol) of PtO₂ were added to a dry flask. 10 mL of methanol were added after the flask was flushed with argon. A solution of 200 mg (0.44 mmol) of ethyl 2(3-hydroxy-4-nitrophenyl)-8,9-dimethoxy-3-methyl-5,6-dihydropyrrolo[2,1-a]isoquinoline-1-carboxylate (Intermediate 6 above) in 10 mL of methanol and tetrahydrofuran (1:1) was added to the flask under argon atmosphere. The solution was degassed and flushed with argon. Hydrogen gas was introduced to the flask by a balloon. The mixture was stirred under hydrogen atmosphere at room temperature overnight. The mixture was filtered and the filtrate was concentrated to give 200 mg of crude, gray product. It was dissolved in 20 mL of trimethyl orthoformate, and the mixture was heated at reflux overnight. Removal of the solvents under reduced pressure gave an oily residue which was purified by HPLC (Method F) to afford 76.6 mg (75%) of the title compound.

MS (HPLC/ES): m/z=433.0 (M+1). LCMS RT (Method G): 3.19 min.

### Example 77

### Ethyl 2-(1,3-benzoxazol-7-yl)-8,9-dimethoxy-3-methyl-5,6-dihydropyrrolo[2,1-a]isoquinoline-1-carboxylate

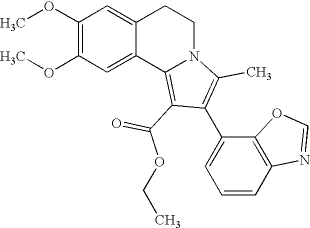

This example was prepared by the method of Example 76 above. MS (HPLC/ES): m/z=433.2 (M+1). HPLC RT (Method G): 2.98 min.

### Example 78

### Ethyl 2-(1-benzyl-2,3-dihydro-1H-indol-5-yl)-8,9-dimethoxy-3-methyl-5,6-dihydropyrrolo[2,1-a]isoquinoline-1-carboxylate

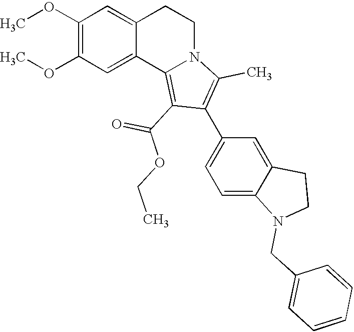

A solution of 3.6 g (15.2 mmol) of Intermediate 7, 1.14 g (15.7 mmol) of nitroethane, 1.55 g (18.2 mmol) of piperidine and 2.10 g (7.59 mmol) of ethyl (2E)-(6,7-dimethoxy-3,4-dihydro-1(2H)-isoquinolinylidene)-ethanoate (Intermediate 2.1) in 35 mL of ethanol and 35 mL of isopropanol was heated at 80° C. overnight. The reaction was then cooled to room temperature and concentrated in vacuo. The crude reddish-orange residue was titurated with refluxing ethyl acetate to afford 2.88 g of the title compound as an off-white solid (5.5 mmol, 72%).

MS (HPLC/ES): m/z=522.9 (M+1). HPLC RT (Method G): 3.84 min.

### Example 79

### Ethyl 2-(2,3-dihydro-1H-indol-5-yl)-8,9-dimethoxy-3-methyl-5,6-dihydropyrrolo-[2,1-a]isoquinoline-1-carboxylate

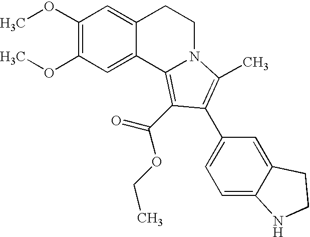

A mixture of 1.00 g (1.91 mmol) of the product obtained in Example 78 and 250.0 mg of palladium hydroxide in 55 mL of dimethyl formamide was stirred for 48 hours under a slight positive pressure of hydrogen. The mixture was filtered through Celite® (diatomaceous earth filter aid, EM Science Brand) to remove the catalyst, and then concentrated in vacuo. The residue was titurated with ether to provide the title compound as a yellow solid 643.1 mg (1.47 mmol, 77% yield).

MS (HPLC/ES): m/z=433.3 (M+1). HPLC RT (Method G): 2.87 min.

### Example 80

### Ethyl 2-{1-[(isopropylamino)-carbonyl]-2,3-dihydro-1H-indol-5-yl}-8,9-dimethoxy-3-methyl-5,6-dihydropyrrolo[2,1-a]isoquinoline-1-carboxylate

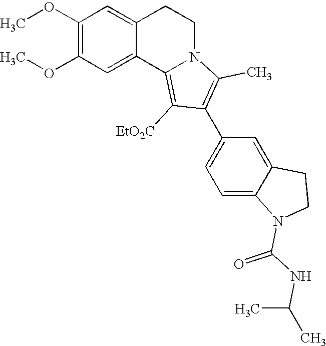

A solution of 75 mg (0.17 mmol) of the product obtained in Example 79 and 15 mg (0.18 mmol) of isopropylisocyanate in 1 mL of dichloromethane was stirred for 18 hours at room temperature, then diluted with 3 mL of hexanes and cooled to −15° C. for 8 hours. Filtration provided the title compound as white crystals 66.2 mg (0.12 mmol, 73%).

MS (HPLC/ES): m/z=518.3 (M+1). HPLC RT(Method G): 3.86 min.

The compounds of the following examples can be prepared using the same method as that employed for the synthesis of Example 80:

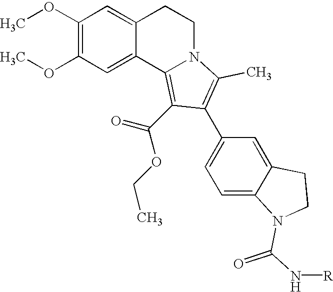

| | | | LC RT |
|---|---|---|---|
| | | HPLC/ES | Method G |
| Example | R | (M + 1) | (min) |
| 81 | 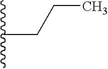 | 518.2 | 3.86 |
| 82 | 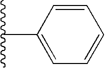 | 552.3 | 4.16 |
| 83 | 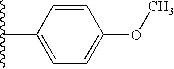 | 582.3 | 4.08 |
| 84 | 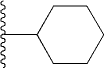 | 558.0 | 3.62 |
| 85 | 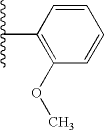 | 581.9 | 3.75 |

### Example 86

### Ethyl 2-(1-acetyl-2,3-dihydro-1H-indol-5-yl)-8,9-dimethoxy-3-methyl-5,6-dihydropyrrolo[2,1-a]isoquinoline-1-carboxylate

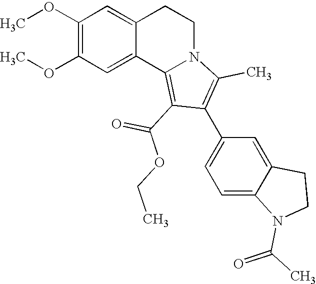

A solution of 55.6 g (0.29 mmol) of the product obtained in Example 79 and 0.060 mL (0.32 mmol) of diisopropylethylamine in 2 mL of dichloromethane was cooled to 0° C. and treated with 0.010 mL (0.16 mmol) of acetyl chloride. The reaction was allowed to stir for 1 hour at 0° C. and then to warm slowly to room temperature over 2 hours, at which time TLC analysis [silica gel 60, ethyl acetate/hexanes (2:3), UV detection] suggested complete reaction. The reaction was diluted with 15 mL of dichloromethane and 15 mL of 1 N phospate buffer. The organic layer was washed with brine, dried over sodium sulfate, filtered and concentrated to give a purple tinted solid. Purification by chromatography [Biotage Flash 21F-SIM, 12 mm×180 cm column, 32-63 μm silica, ethyl acetate/hexane (35:65)] gave the title compound as an off-white solid 37.2 mg (61%).

MS (HPLC/ES): m/z=475.2 (M+1). HPLC RT (Method G): 3.67.

The compounds of the following examples can be prepared using the same method as that employed for the synthesis of Example 86:

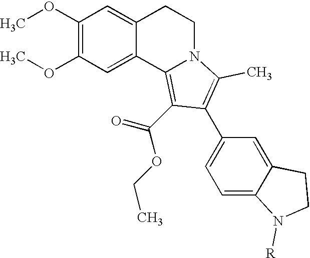

| | | | LCRT |
|---|---|---|---|
| | | HPLC/ES | Method G |
| Example | R | (M + 1) | (min) |
| 87 | 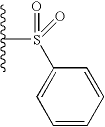 | 595.2 (M + Na) | 4.27 |
| 88 | 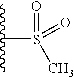 | 510.9 | 3.26 |
| 89 | 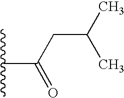 | 517.3 | 4.14 |
| 90 | 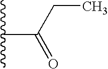 | 489.3 | 3.82 |
| 91 | 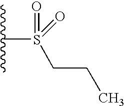 | 539.1 | 3.41 |
| 92 | 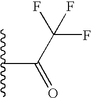 | 529.0 | 3.71 |

We claim:

1. A compound of the formula 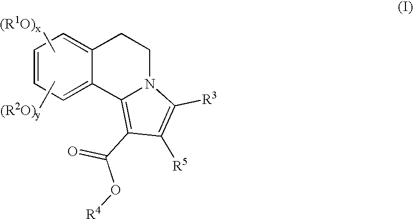
wherein
x and y independently from each other denote zero or 1;
R¹ and R² independently from each other denote hydrogen, C₁₋₄-alkyl or CF₃;
R³ and R⁴ independently from each other denote C₁₋₄-alkyl;
R⁵ denotes
i) C₁₋₁₂-alkyl, optionally having from 1 to 3 substituents selected from the group consisting of C-₁₋₆-alkoxy, C₆₋₁₀-aryl, and heteroaryl;
or
ii) C₃₋₈-cycloalkyl, optionally having from 1 to 3 substituents selected from the group consisting of C₁₋₆-alkyl, C-₁₋₆-alkoxy, COOR⁶, C₆₋₁₀-aryl, and heteroaryl;
or
iii) heteroaryl optionally substituted with up to 3 substituents selected from the group consisting of
a) C₁₋₆-alkyl, C-₁₋₆-alkoxy, C₆₋₁₀-aryl-C₁₋₆-alkyl, heteroaryl-C₁₋₆-alkyl, C₁₋₆-alkoxy-C₁₋₆-alkyl, C₁₋₆-alkoxy-C₁₋₆-alkoxy-C₁₋₆-alkyl, cyano-C₁₋₆-alkyl, and C₆₋₁₀-aryl (each of which can optionally be substituted by halogen up to perhalo),
b) COR⁶,
c) COOR⁶,
d) hydroxyl,
e) halogen,
f) cyano,
g) SO₂R⁶, and
h) saturated 5- to 9-membered nitrogen-containing heterocyclyl (which saturated heterocyclyl may contain up to 2 further heteroatoms selected from the group consisting of N, O and S and which saturated heterocyclyl can be further substituted with one or more radicals selected from the group consisting of hydroxyl, NH₂, C₁₋₆-alkyl, C₁₋₆-alkoxy, and C₆₋₁₀-aryl);
wherein R⁶ denotes
1) hydrogen,
2) C₁₋₆-alkyl optionally substituted with halogen up to perhalo,
3) C₃₋₈-cycloalkyl,
4) C₆₋₁₀-aryl optionally substituted with C₁₋₆-alkoxy,
5) heteroaryl-C₁₋₆-alkyl,
6) C₆₋₁₀-aryl-C₁₋₆-alkyl optionally substituted with up to 2 C₁₋₆-alkoxy, or
7) —NR⁷R⁸, wherein (i) R⁷ and R⁸ are each independently selected from the group consisting of hydrogen, C₁₋₆-alkyl, C₃₋₈-cycloalkyl, heterocyclyl, and C₆₋₁₀-aryl optionally substituted with C₁₋₆-alkoxy, or
(ii) R⁷ and R⁸ together with the nitrogen atom to which they are attached form a 5- to 7-membered heterocyclyl which may contain up to 2 further hetero atoms selected from the group consisting of N, O, and S, which heterocyclyl can further be substituted with 1 to 3 radicals selected from the group consisting of OH, C₁₋₄-alkyl, C₁₋₄-alkoxy, C₆₋₁₀-aryl, and heteroaryl;
or
iv) phenyl fused to a 5- to 7-membered saturated cycloalkyl optionally containing up to two heteroatoms selected from the group consisting of O, N, and S, with the proviso that both heteroatoms cannot be O, optionally substituted with 1-3 substituents selected from the group consisting of hydroxy, halogen, C₁₋₆-alkyl, C₁₋₆-alkoxy, C₁₋₆-alkylsulfonyl, phenylsulfonyl, N-C₁₋₆-alkylcarboxamido, N-(C₃₋₈-cycloalkyl)-carboxamido, N-phenylcarboxamido, N-(C₁₋₆-alkoxyphenyl)-carboxamido; and (C₁₋₆-alkyl)-carbonyl, which (C₁₋₆-alkyl)-carbonyl may optionally be substituted by halogen up to perhalo,
and an isomer, a pharmaceutically acceptable salt, a hydrate, or a hydrate of a pharmaceutically acceptable salt thereof.

2. A compound according to claim 1, wherein
x and y independently from each other denote zero or 1;
R¹ and R² independently from each other denote hydrogen, C₁₋₄-alkyl or CF₃;
R³ and R⁴ independently from each other denote C₁₋₄-alkyl;
R⁵ denotes
i) C₁₋₁₂-alkyl, optionally having 1 to 3 substituents selected from the group consisting of C-₁₋₆-alkoxy and C₆₋₁₀-aryl;
or
ii) C₃₋₈-cycloalkyl;
or
iii) heteroaryl optionally substituted with up to 3 substituents selected from the group consisting of
a) C₁₋₆-alkyl, C-₁₋₆-alkoxy, C₆₋₁₀-aryl-C₁₋₆-alkyl, heteroaryl-C₁₋₆-alkyl, C₁₋₆-alkoxy-C₁₋₆-alkyl, C₁₋₆-alkoxy-C₁₋₆-alkoxy-C-₁₋₆-alkyl, cyano-C₁₋₆-alkyl, and C₆₋₁₀-aryl (each of which can optionally be substituted with halogen radicals up to perhalo),
b) COR⁶,
c) COOR⁶,
d) hydroxyl,
e) halogen,
f) cyano,
g) SO₂R⁶, and
h) saturated 5- to 9-membered nitrogen-containing heterocyclyl (which saturated heterocyclyl may contain up to 2 further hetero atoms selected from the group consisting of N, O and S and which saturated heterocyclyl can be further substituted with one or more radicals selected from the group consisting of hydroxyl, NH₂, C₁₋₆ alkyl, C₁₋₆-alkoxy, and C₆₋₁₀-aryl);
wherein R⁶ denotes
1) hydrogen,
2) C₁₋₆-alkyl optionally substituted with halogen up to perhalo,
3) C₃₋₈-cycloalkyl,
4) C₆₋₁₀-aryl optionally substituted with C₁₋₆-alkoxy,
5) heteroaryl-C₁₋₆-alkyl,
6) C₆₋₁₀-aryl-C₁₋₆-alkyl optionally substituted with up to 2 C₁₋₆-alkoxy, or
7) —NR⁷R⁸ wherein R⁷ and R⁸ are each independently selected from the group consisting of hydrogen, C₁₋₆-alkyl, C₃₋₈-cycloalkyl, heterocyclyl, or C₆₋₁₀-aryl which C₆₋₁₀-aryl is optionally substituted with C₁₋₆-alkoxy;
or
iv) indolinyl optionally substituted with up to three substituents selected from the group consisting of hydroxy, halogen, C₁₋₆-alkyl, C₁₋₆-alkoxy, C₁₋₆-alkylsulfonyl, phenylsulfonyl, N-C₁₋₆-alkylcarboxamido, N-(C₃₋₈-cycloalkyl)-carboxamido, N-phenylcarboxamido, N-(methoxyphenyl)-carboxamido, and (C₁₋₆-alkyl)-carbonyl wherein said (C₁₋₆)-alkyl)carbonyl may optionally be substituted by halogen up to perhalo,
and an isomer, a pharmaceutically acceptable salt, a hydrate, or a hydrate of a pharmaceutically acceptable salt thereof.

3. A compound according to claim 1, wherein
x and y denote each 1;
R¹ and R² independently from each other denote hydrogen or C₁₋₄-alkyl;
R³ and R⁴ independently from each other denote C₁₋₄-alkyl;
R⁵ denotes
i) methyl, ethyl, n-propyl, iso-propyl, n-butyl, n-pentyl, n-hexyl, n-heptyl, 2-phenylpropyl, 2-butyl, benzyl;
or
ii) cyclopropyl, cyclopentyl or cyclohexyl;
or
iii) a) thienyl optionally substituted with Cl, Br, I, C₁₋₆-alkyl;
b) pyrrolyl optionally substituted with Cl, Br, I, C₁₋₆-alkyl;
c) furyl optionally substituted with Cl, Br, I, C₁₋₆-alkyl;
d) thiazolyl optionally substituted with Cl, Br, I, C₁₋₆-alkyl;
e) imidazolyl optionally substituted with Cl, Br, I, C₁₋₆-alkyl;
f) pyridyl optionally substituted with Cl, Br, I;
g) pyrimidinyl optionally substituted with pyrrolidine;
h) indazolyl optionally substituted with 2-fluorobenzyl;
i) benzimidazolyl;
j) benzoxazolyl;
k) quinolyl optionally substituted with hydroxyl, methyl or phenyl; or
l) indolyl optionally substituted with up to three substituents selected from the group consisting of F, Cl, Br, I, C₁₋₆-alkyl, C₁₋₆-alkoxy-C₁₋₆-alkyl, C₁₋₆-alkoxy-C₁₋₆-alkoxy-C₁₋₆-alkyl, cyano-C₁₋₆-alkyl, C₁₋₆-alkoxy, benzyl, fluorobenzyl, pyridylmethyl, phenylsulfonyl, formyl, (C₁₋₆-alkyl)-carbonyl, (C₃₋₈-cycloalkyl)-carbonyl, phenylcarbonyl, methoxyphenylcarbonyl, and dimethoxybenzylcarbonyl;
or
(iv) indolinyl optionally substituted with up to three substituents selected from the group consisting of C₁₋₆-alkylsulfonyl, phenylsulfonyl, N-C₁₋₆-alkylcarboxamido, N-(C₃₋₈-cycloalkyl)-carboxamido, N-phenylcarboxamido, N-(methoxyphenyl)-carboxamido, and (C₁₋₆-alkyl)-carbonyl wherein said (C₁₋₆-alkyl)-carbonyl may optionally be substituted by halogen up to perhalo,
and an isomer, a pharmaceutically acceptable salt, a hydrate, or a hydrate of a pharmaceutically acceptable salt thereof.

4. A compound according to claim 3 of the formula: 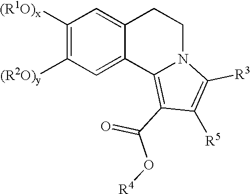
wherein x, y, R¹, R², R³, R⁴, and R⁵ are as defined in claim 3.

5. A compound according to claim 4, wherein said compound is selected from the group consisting of: 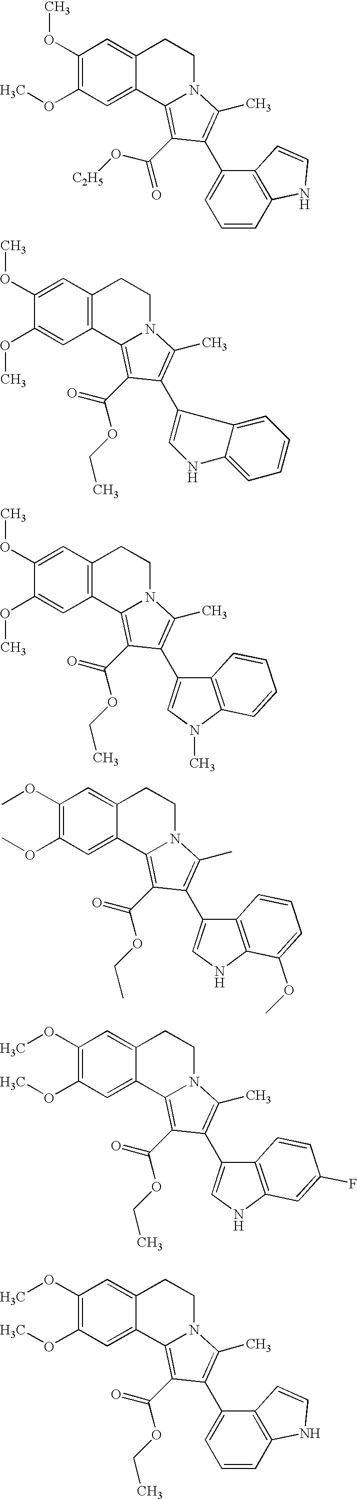 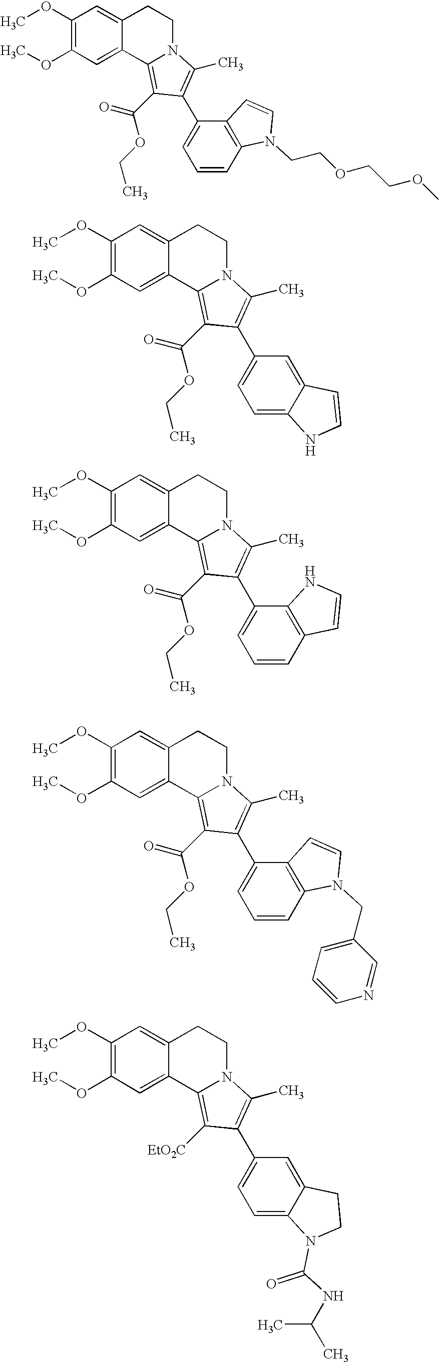 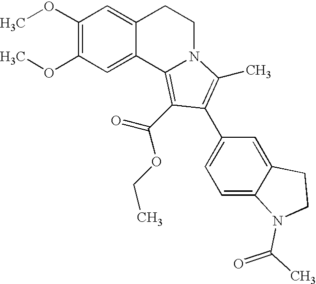
and an isomer, a pharmaceutically acceptable salt, a hydrate, or a hydrate of a pharmaceutically acceptable salt thereof.

6. A process for manufacturing a compound of claims 1 to 5, comprising the reaction of a compound of the formula 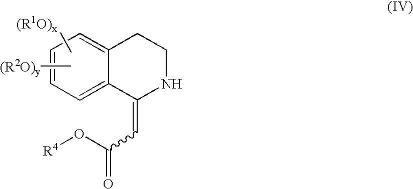
wherein x, y, R¹, R² and R⁴ are as defined in claims 1 to 5,
[A] with the compounds of the formulae
R⁵—CHO (II) and R³—CH₂—NO₂ (III)
wherein R³ and R⁵ are as defined in claims 1 to 5, or
[B] with a compound of the formula 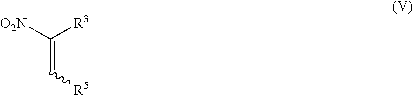
wherein R³ and R⁵ are as defined in claims 1 to 5,
and optionally
[C] conversion of the compound obtained through either process [A] or [B] into an isomer, a pharmaceutically acceptable salt, a hydrate, or a hydrate of a pharmaceutically acceptable salt thereof.

7. A compound according to claims 1 to 5 for use in a medicinal application.

8. A compound according to claims 1 to 5 for combating cancer.

9. A method of manufacturing a pharmaceutical composition by combining at least one of the compounds of claims 1 to 5 with at least one pharmacologically acceptable formulating agent.

10. A pharmaceutical composition comprising as an active ingredient an effective amount of at least one of the compounds of claims 1 to 5 and at least one pharmacologically acceptable formulating agent.

11. A pharmaceutical composition comprising as an active ingredient as effective amount of at least one of the compounds of claims 1 to 5 and at least one pharmaceutically active ingredient which is different from the compounds of claims 1 to 5.

12. A method of combating cancer in humans and animals comprising the administration of an effective amount of at least one compound of claims 1 to 5.

13. A medicament in unit dosage form comprising an effective amount of a compound of claims 1 to 5 together with an inert pharmaceutical carrier.

14. Use of at least one of the compounds of claims 1 to 5 for manufacture of a medicament for combating cancer.
